(19) United States
(12) Patent Application Publication
(10) Pub. No.: US 2026/0015579 A1
(43) Pub. Date: Jan. 15, 2026

(54) ARTIFICIAL UTERINE PLATFORM AND APPLICATIONS THEREOF

(71) Applicants: California Institute of Technology, Pasadena, CA (US); The Regents of the University of California, Oakland, CA (US)

(72) Inventors: Magdalena D. Zernicka-Goetz, Pasadena, CA (US); Mohammad Ikbal B. Choudhury, Pasadena, CA (US); Christopher C. W. Hughes, Irvine, CA (US); Makena Ewald, Irvine, CA (US)

(21) Appl. No.: 19/267,498

(22) Filed: Jul. 12, 2025

Related U.S. Application Data
(60) Provisional application No. 63/670,520, filed on Jul. 12, 2024.

(51) Int. Cl. C12N 5/073; C12M 1/00; C12M 1/12; C12M 3/06; C12N 5/00; C12N 5/071

(52) U.S. Cl. CPC C12N 5/0604; C12M 23/16; C12M 25/14; C12M 29/10; C12N 5/0018; C12N 5/0697; C12N 2500/02; C12N 2500/32; C12N 2500/38; C12N 2500/44; C12N 2500/84; C12N 2501/01; C12N 2501/105; C12N 2501/11; C12N 2501/115; C12N 2501/165; C12N 2501/392; C12N 2501/91; C12N 2502/02; C12N 2502/1323; C12N 2502/27; C12N 2521/00; C12N 2533/52; C12N 2533/54; C12N 2533/56; C12N 2537/10; C12N 2539/00

(57) ABSTRACT

Provided herein include synthetic endometrial tissues and uterus-like structures for assessing embryo implantation and growth in vitro. Provided herein also include methods and devices for making the synthetic endometrial tissues and uterus-like structures and uses thereof to culture embryos (e.g., human embryos). The methods and synthetic endometrial tissues and structures generated herein can be used to culture pre-implantation embryos (e.g., human embryos) to reach a post-implantation stage.

### RELATED APPLICATIONS

This application claims the benefit under 35 U.S.C. § 119 (e) of U.S. Provisional Patent Application Ser. No. 63/670,520, filed on Jul. 12, 2024, the content of this related application is incorporated herein by reference in its entirety for all purposes.

### BACKGROUND

### Field

The present disclosure relates generally to the field of embryo development and synthesis.

### Description of the Related Art

Human reproduction is remarkably inefficient, in comparison to other mammalian species, because the majority of fertilization events do not culminate in a live birth. Infertility affects one in five couples, and its incidence continues to rise, with increasing numbers of couples turning to assisted reproductive technologies (ART). Much research on ART has focused on the embryonic contribution to implantation, including aneuploidy and embryo quality. However, at best, only a 60-70% pregnancy rate can be achieved with in vitro fertilization, even when selecting only the best quality euploid embryos for transfer. This reflects the fact that embryo implantation requires a highly coordinated series of both embryonic and maternal events involving embryo-endometrial crosstalk, endometrial receptivity, and immunological tolerance. The exact reason that leads to implantation failure remains a mystery. There remains a need to develop in vitro platform and protocols that can be used to assess the implantation and growth of mammalian embryos and stem cell-based embryo models.

### SUMMARY

Provided herein includes a method for preparing a perfusable vascularized tissue for in vitro embryo implantation and growth. The method comprises co-culturing endothelial cells and fibroblast cells in a hydrogel under a condition allowing the endothelial cells and fibroblast cells to self-organize into a perfusable vascularized tissue.

In some embodiments, the endothelial cells comprise endothelial progenitor cells derived from umbilical cord blood, human umbilical cord vascular endothelial cells, mouse brain endothelial cells, an immortalized vascular endothelial cell line, or any combination thereof. The immortalized vascular endothelial cell line can be selected from the group consisting of: EA.hy926, HMEC-1, TIME cells, CI-huMEC, EC-RF24, iMAEC, or a combination thereof. The fibroblast cells can comprise human uterine fibroblast cells, human primary brain vascular fibroblast, mouse embryonic brain vascular fibroblast, human dermal fibroblast, human perivascular fibroblast, human lung fibroblast, or any combination thereof. In some embodiments, the endothelial cells comprise human umbilical cord vascular endothelial cells and/or the fibroblast cells comprise human lung fibroblast cells.

In some embodiments, the hydrogel is a natural hydrogel or a synthetic hydrogel. The hydrogel can comprise collagen, hyaluronic acid, polyethylene glycol, polyacrylamide, or a combination thereof. In some embodiments, the hydrogel is fibrin, optionally the fibrin is fibrinogen cross-linked with thrombin at a thrombin: fibrinogen ratio of about 1:5. In some embodiments, the hydrogel is functionalized with one or more extracellular matrix component, optionally the extracellular matrix component comprises laminin, fibronectin, collagen-IV, collagen-I, hyaluronic acid, or any combination thereof. The hydrogel can comprise about 60% fibrin, about 20% collagen-I, about 10% laminin, and about 10% fibronectin, optionally, the fibrin comprises thrombin and fibrinogen at a ratio of about 1:5.

In some embodiments, the culturing comprises adding a laminin solution to a solidified hydrogel mixture comprising the endothelial cells and fibroblast cells and incubating for a time period, optionally the incubation is for about 15 minutes, and replacing the laminin solution with a vasculature media, optionally, the vasculature media is endothelial cell growth medium. A hydrostatic gradient of the vasculature media can be maintained to generate a media flow from a higher pressure area to a lower pressure area. In some embodiments, the vasculature media comprises fetal bovine serum, hydrocortisone, human fibroblast growth factor-B (hFGF-B), vascular endothelial growth factor (VEGF), R3-insulin-like growth factor-1 (R3-IGF-1), ascorbic acid, heparin, human epidermal growth factor (hEGF), an antimicrobial, or any combination thereof.

In some embodiments, the co-culturing occurs in a microfluidic device. The microfluidic device can comprise at least one tissue chamber, wherein the at least one tissue chamber comprises an enclosed chamber, an exposed chamber accessible from a top plate via an opening, and interdigitated pillars at the interface between the enclosed chamber and the exposed chamber, the enclosed chamber in fluidic communication with the enclosed chamber, and wherein the enclosed chamber is fluidly connected to a media inlet and a media outlet, and the enclosed chamber and the exposed chamber each is connected to at least one hydrogel loading port. The method can further comprise seeding the endothelial cells, the fibroblast cells, and the hydrogel in the enclosed chamber and in the exposed chamber via different ports, thereby creating a continuous solution in both the enclosed and exposed chambers. Seeding the endothelial cells, the fibroblast cells, and the hydrogel in the enclosed chamber and in the exposed chamber can occur sequentially, optionally, seeding the endothelial cells, the fibroblast cells, and the hydrogel in the exposed chamber follows seeding the endothelial cells, the fibroblast cells, and the hydrogel in the enclosed chamber. The method can further comprise adding the laminin solution and/or the vasculature media to the solidified hydrogel mixture via the media inlet and/or the media outlet. In some embodiments, the co-culturing is for at least about 5-6 days or until the vasculature fuses with microfluidic channels to create a perfusable network.

Provided herein also includes a method of preparing a uterus-like tissue for in vitro embryo implantation and culturing. The method can comprise preparing a perfusable vascularized tissue using the method described herein, priming endometrial epithelial organoids and stromal cells with one or more hormones, and assembling the primed endometrial epithelial organoids and primed stromal cells with the perfusable vascularized tissue to form a uterus-like tissue. The one or more hormones can comprise β-estradiol (E2), Medroxyprogesterone acetate (MPA), Progesterone (P4), cyclic adenosine monophosphate (cAMP), or any combination thereof. In some embodiments, the endometrial epithelial organoids and the stromal cells are primed separately. The assembling can comprise culturing the primed endometrial epithelial organoids and primed stromal cells in the perfusable vascularized tissue in a minimal differentiation media in the presence of the one or more hormones. In some embodiments, the minimal differentiation media comprises a basal culture media supplemented with N-acetylcysteine, eestradiol, a cyclic AMP analogue, optionally, 8-bromo-cAMP, and a progestin, optionally, medroxyprogesterone acetate (MPA). The hydrogel composition can comprise about 60% fibrin, about 20% collagen-I, about 10% laminin, and about 10% fibronectin. The concentration of the stromal cells can be about 1.5 million/mL.

In some embodiments, the assembling occurs in a microfluidic device comprising at least one tissue chamber, wherein the at least one tissue chamber comprises an enclosed chamber, an exposed chamber accessible from a top plate via an opening, and interdigitated pillars at the interface between the enclosed chamber and the exposed chamber, the enclosed chamber in fluidic communication with the enclosed chamber, and wherein the enclosed chamber is fluidly connected to a media inlet and a media outlet, and the enclosed chamber and the exposed chamber each is connected to at least one hydrogel loading port. The method can further comprise loading the primed endometrial epithelial organoids and primed stromal cells to the tissue chamber containing the perfusable vascularized tissue, optionally the loading occurs through the opening in the exposed chamber.

Provided herein also includes a perfusable vascularized tissue and a uterus-like tissue for in vitro embryo implantation (e.g., human embryo implantation) and culturing obtainable by any one of the methods disclosed herein.

Provided herein also includes a method of culturing an embryo (e.g., a mammalian embryo such as a human embryo) in vitro. In some embodiments, the method comprises preparing a perfusable vascularized tissue according to the method described herein, and culturing an embryo at pre-implantation stage in the perfusable vascularized tissue in a culture media under a condition allowing the embryo to reach a post-implantation stage. In some embodiments, the method comprises preparing a uterus-like tissue structure according to any one of the methods described herein, and culturing an embryo at pre-implantation stage in the uterus-like tissue structure in a culture media under a condition allowing the embryo to reach a post-implantation stage. The embryo disclosed herein can be a mammalian embryo (e.g., a human embryo). In some embodiments, the mammalian embryo is at day 5 after fertilization or at Carnegie stage 3-2 prior to culturing in the perfusable vasculature tissue. In some embodiments, the human embryo at pre-implantation stage is at a blastocyst stage. In some embodiments, the mammalian embryo is a non-human embryo such as a mouse embryo, a rabbit embryo or a bovine embryo. In some embodiments, the mammalian embryo is a stem-cell derived embryo model (for example, a blastoid). In some embodiments, the mammalian embryo is a human embryo.

In some embodiments, culturing the embryo in the perfusable vascularized tissue or in the uterus-like tissue structure comprises transferring the embryo to the perfusable vascularized tissue or the uterus-like tissue structure and embedding the embryo in the perfusable vascularized tissue or the uterus-like tissue structure. Transferring the embryo to the perfusable vascularized tissue can comprise placing the embryo on the top of the perfusable vascularized tissue. Transferring and embedding the embryo can occur concurrently or sequentially. In some embodiments, embedding the embryo in the perfusable vascularized tissue or the uterus-like tissue structure occurs at least two days (e.g., two days, three days, or more) after transferring the embryo to the perfusable vascularized tissue or the uterus-like tissue structure or upon collapse of the blastocoel cavity of the embryo. In some embodiments, transferring the embryo to the perfusable vascularized tissue or the uterus-like tissue structure and embedding the embryo in the perfusable vascularized tissue or the uterus-like tissue structure occurs at the same day, optionally at day 6 after fertilization. The embryo disclosed herein can be a mammalian embryo (e.g., a human embryo).

In some embodiments, the embryo is cultured under a dynamic condition comprising a progressive increase of oxygen concentration and/or gas pressure. The oxygen concentration can be increased from about 5% to about 12%. The gas pressure can be increased from about 2 psi to about 5 psi. In some embodiments, the oxygen concentration is increased by 1% daily and/or the gas pressure is increased by 0.5 psi daily. In some embodiments, the culture media comprises a basal culture medium with a progressive increase in fetal bovine serum from about 10% to about 30%. The fetal bovine serum can be maintained at about 10% for two days, at about 20% for two days, and at about 30% for two days. The culture media can comprise DMEM/F12, neurobasal A, B27, N2, GMAX, non-essential amino acids, penicillin/streptomycin, beta-mercaptoethanol, sodium pyruvate, sodium lactate, hlGF1, β-estradiol, progesterone, N-acetylcysteine, or any combination thereof. The embryo can be cultured in a global culture media prior to culturing in the perfusable vascularized tissue, optionally for 1, 2, 3, 4, or 5 days, and optionally wherein the oxygen concentration is maintained at about 5% and the gas pressure at about 2 psi, and optionally the global culture media comprises at least 5% human serum albumin.

In some embodiments, the perfusable vascularized tissue is supplied with a vasculature media prior to culturing the embryo. The vasculature media can comprise fetal bovine serum, hydrocortisone, human fibroblast growth factor-B (hFGF-B), vascular endothelial growth factor (VEGF), R3-insulin-like growth factor-1 (R3-IGF-1), ascorbic acid, heparin, human epidermal growth factor (hEGF), an antimicrobial, or any combination thereof. In some embodiments, the uterus-like tissue is supplied with a vasculature media (e.g., a minimum differentiation media) prior to culturing the embryo. The minimal differentiation media can comprise a basal culture media supplemented with N-acetylcysteine, estradiol, a cyclic AMP analogue, optionally, 8-bromo-cAMP, and a progestin, optionally, medroxyprogesterone acetate (MPA).

In some embodiments, the embryo is cultured in the perfusable vascularized tissue or the uterus-like tissue structure for at least 6 days (e.g., at least 7, 8, 9, 10, 11, 12, 13, 14, 15, 16, 17, 18, 19, 20, or more days) and/or reaches at least Carnegie stage 5c (e.g., reaches to term). The embryo at the post-implantation stage can comprise NANOG+ epiblast cells cavitating to give rise to an amniotic cavity, SOX17+ hypoblast cells forming a primary yolk sac, and AP2-Gamma (AP2Y)+ trophoblast cells forming a trophoblast region. In some embodiments, the mammalian embryo at the post-implantation stage comprises primary yolk sac with SOX17+ visceral and parietal endoderm cells, a chorionic cavity, NANOG+SOX17+AP2-gamma+ primordial germ cells, and/or migratory trophoblast cells that interact with endothelial vessels to cause vascular mimicry, vasodilation and angiogenesis. In some embodiments, the mammalian embryo is a blastoid and the morphogenesis of the mammalian embryo at the post-implantation stage resembles that of a natural embryo. In some embodiments, the embryo culture media and/or the vasculature media is positive for human chorionic gonadotropin (hCG), a biomarker to detect pregnancy in humans, following the culturing of the mammalian embryo. In some embodiments, culturing the embryo takes place in a microfluidic device. The embryo disclosed herein can be a human embryo and human embryo model.

Provided herein also includes a microfluidic platform. In some embodiments, the microfluidic platform comprises a plurality of microfluidic devices or bioreators each comprising at least one tissue chamber, wherein the at least one tissue chamber comprises an enclosed chamber, an exposed chamber accessible from a top plate via an opening, and interdigitated pillars at the interface between the enclosed chamber and the exposed chamber, the enclosed chamber in fluidic communication with the enclosed chamber, and wherein the enclosed chamber is fluidly connected to a media inlet and a media outlet, and the enclosed chamber and the exposed chamber each is connected to at least one hydrogel loading port.

In some embodiments, the enclosed chamber comprises at least two perfusion microvalves connecting the enclosed chamber to microfluidic channels located parallel to the enclosed chamber. The at least two perfusion microvalves can have a width of about 50 μm to about 100 μm. The at least two perfusion microvalves each can have a burst pressure and is configured to have a first function of preventing hydrogel or hydrogel precursor from bursting into at least one of the microfluidic channels and a second function for perfusing nutrients through the tissue chamber. The media inlet can be connected to the enclosed chamber through one perfusion microvalve and the media outlet can be connected to the enclosed chamber through another perfusion microvalve. The exposed chamber can be configured for loading embryos and embryo culture media from the opening. The media inlet and/or outlet can be configured for loading vasculature media. The at least one hydrogel loading port can be configured for loading hydrogel, hydrogel precursor, or a cell-hydrogel solution. In some embodiments, the enclosed chamber and the exposed chamber are seeded with endothelial cells, fibroblast cells, and a hydrogel for preparing a perfusable vascularized tissue. The hydrogel can be collagen-base hydrogel, fibrin-based hydrogel, poly(ethylene glycol)-based hydrogel, or a combination thereof. In some embodiments, the enclosed chamber and the exposed chamber are connected to different hydrogel loading ports.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D depict an exemplary microfluidic vasculature-on-a-chip used herein for preparing perfusable vasculature. FIG. 1A: 96 well plate setup for that houses 16 devices with perfuseable blood vessels. FIG. 1B: Schematic of the tissue chamber used to seed endothelial and fibroblast cells in a fibrin-based hydrogel. The tissue chamber is composed of two parts: enclosed and exposed (punch out). The exposed part is open and is used for implantation of the embryo. The tissue chamber is connected to the media inlet and outlet channels via two ports.

FIG. 1C: Schematic of the microfluidic channels that enable perfusion of fluid into and out of the tissue chamber. A fluid pressure gradient is created between the M1 and M2 ports to drive fluid flow. FIG. 1D. 3D reconstruction of the tissue chamber along with media inlet and outlet channels.

FIG. 2 displays non-limiting exemplary timelines of vascular self-organization. A. endothelial (Green) and fibroblast (blue) cells are mixed in the hydrogel at a concentration of 3 Million/mL and seeded into the tissue chamber. Both cell types proliferate and self-organize with a few days to give rise to a closed-loop vasculature within the hydrogel.

FIGS. 3A-3B display generation of a perfuseable vascular network. The endothelial vessels anastomose into the media channels from Day 3 to generate a perfusable network, where media can flow in and out between microfluidic channels and the blood vessels. Vessels start maturing by Day 5 and become tight (leakage free) by Day 7. A 70 kDa FITC dextran dye (Green) is used to visualize perfusion of the dye and the vasculature. Endothelial cells are labelled in red.

FIG. 4 depicts an exemplary protocol of embryo implantation in the vascularized device. A detailed protocol with timeline for culturing the embryo is shown. The protocol is divided into two phases: generation of perfuseable vasculature and culture of the embryo. During the vasculature phase, oxygen tension is maintained at 5% at 2 psi gas pressure and during the embryo culture phase, both oxygen tension and gas pressure are dynamically increased from 5% at 2 psi till 12% at 5 psi. Media for the embryo is dynamically changed as shown.

FIG. 5 depicts a breakup of embryo media components and incubation parameters along the timeline.

FIGS. 6A-6B display an exemplary dissection of tissue out of the microfluidic device. FIG. 6A shows region for punch out (in red dashed circle) of the microfluidic device. The tissue is then dissected out using forceps under a microscope. FIG. 6B shows an image of the vascularized tissue with embedded embryo under a brightfield microscope.

FIGS. 7A-7D display non-limiting exemplary immunofluorescence images of cryosections of four embryos cultured in the vascularized device till Day 10 (10 dpf). NANOG+ cells indicate epiblast, SOX17+ cells indicate hypoblast, AP2-Gamma+ (TFAP2C) cells indicate trophoblast and CD31+ (PECAM) cells indicate endothelial blood vessels.

FIGS. 8A-8C display non-limiting exemplary montage of immunofluorescence (IF) images of different sections of the sample containing Embryo #2 is shown. Images indicate changes in morphology of NANOG+ epiblast cells from pseudostratfied to squamous around the amniontic cavity.

FIGS. 9A-9B display non-limiting exemplary immunofluorescence images showing hypoblast differentiation and the primary yolk sac. D10 embryo #9 and Embryo #7 developed Primary Yolk Sac (PYS) enclosed by the visceral endoderm (VE) adjacent to the epiblast and parietal endoderm (PE). Visceral endoderm cells have higher density and exhibits cuboidal morphology compared to the parietal endoderm cells which are more squamous in morphology. NANOG+ cells indicate epiblast, SOX17+ cells indicate hypoblast, AP2-Gamma+ (TFAP2C) cells indicate trophoblast, and CD31+ (PECAM) cells indicate endothelial blood vessels.

FIGS. 10A-10D display non-limiting exemplary immunofluorescence images showing morphogenesis of primary yolk sac (PYS). Analysis of D10 embryo #7 reveals splitting of the parietal endoderm into small vesicles. Analysis of Embryo #2 and Embryo #13 shows detachment of PYS from the trophoblast tissue as indicated by arrowheads.

FIGS. 11A-11C display non-limiting exemplary immunofluorescence images showing amnion and primordia germ cells (PGCs) specification. By D12 (12 dpf) NANOG+epiblast cells gave rise to the extra-embryonic amnionic ectoderm (or Amnion) cells, which have a distinct squamous epithelial morphology compared to the columnar cuboidal morphology of epiblast cells. The epiblast cells also gives rise to the PGCs as indicated by the combined expression of NANOG+SOX17+AP2Y.

FIGS. 12A-12C display non-limiting exemplary immunofluorescence images showing differentiation and morphogenesis of trophoblast cells. FIG. 12A: The trophoblast cells (AP2Y+) were in close proximity to the vasculature (CD31+). FIG. 12B: Chorionic activity. FIG. 12C: EVT-like cells can be visible migrating away from the embryo towards the blood vessels.

FIGS. 13A-13C display non-limiting exemplary immunofluorescence images showing trophoblast-vascular interaction. FIG. 13A: By Day12, blood vessels around the embryo enlarged. FIG. 13B: Magnified view of the left region on interest shows that trophoblast cells (AP2Y+) cells in close proximity around a blood vessel on the other side of the embryo. FIG. 13C: Magnified view of the right region on interest on FIG. 13A shows trophoblast cells forming a blood vessel sharing CD31 membrane marker. MaxP means maximum projection image. Scale bar=50 μm.

FIGS. 14A-14C display non-limiting exemplary immunofluorescence images showing trophoblast-vascular interaction. FIG. 14A: Maximum projection image shows AP2Y+ trophoblast cells interacting with CD31+ endothelial vessel. FIG. 14B: Z-slice1 shows a trophoblast cell positioned behind an endothelial cell forming the blood vessel. FIG. 14C: Z-Slice2 shows another trophoblast cell acquiring replaced endothelial cells to become part of the vascular lining indicating vascular mimicry. Arrow heads indicate location of trophoblast cells. FIG. 14D: Absolute values of HCG measured in the vascular media at different timepoints of human embryo culture in the vascularized platform.

FIGS. 15A-15C display non-limiting exemplary immunofluorescence images showing 3D embedding embryos on D6. FIG. 15D depicts the overview of an exemplary protocol showing transfer and embedding embryo on Day 6.

FIGS. 16A-16C display non-limiting exemplary immunofluorescence images showing that 3D embedding of embryo on D6 leads to decreased hypoblast cells and PYS. FIG. 16A: Schematic representation of embryo encapsulated adjacent to pre-formed vasculature. FIG. 16B: Image shows 3D view of the encapsulated embryo with NANOG+ epiblast, SOX17+ hypoblast and AP2Y+Trophoblast cells. FIG. 16C: Two slices indicating different sections of the embryo and amniotic cavity (AC). Arrowheads indicate epiblast cells with increased expression of AP2Y.

FIG. 17 displays non-limiting exemplary immunofluorescence images demonstrating that two z-slices of the embryo shows differentiation of trophoblast cells into cytotrophoblast (CTB) and syncytiotrophoblast (STB) cells in close proximity.

FIGS. 18A-18B display non-limiting exemplary immunofluorescence images related to two z-slices of the embryo showing trophoblast-vascular interaction.

FIGS. 19A-19C display non-limiting exemplary immunofluorescence images showing blastoids derived from naïve stem cells. FIG. 19A: Protocol for generation of human stem-cell derived embryo model termed “Blastoid”. FIG. 19B: A collection of Day 4 blastoids immunostained for epiblast (NANOG), Hypoblast (SOX17) and Trophoblast (AP2Y). FIG. 19C: Magnified view of a single blastoid.

FIGS. 20A-20B display non-limiting exemplary images of blastoides embedded on D4. Blastoids implanted in the device develop a primary yolk-sac lined by hypoblast cells (Sox17+). The hypoblast cells in proximity to the epiblast are termed as visceral endoderm (VE) and the hypoblast in proximity to trophoblast cells are termed as parietal endoderm (PE) cells. The AP2Y+ trophectoderm cells have differentiated into a thicker layer of cells. F-actin staining indicates cell surface and cavities.

FIG. 21 displays non-limiting exemplary immunofluorescence images showing the cross section of D7 blastoid indicating, epiblast, ITGA+ cytotrophoblast (CTB) and SDC1+ syncytiotrophoblast (STB) cells.

FIG. 22 displays a non-limiting exemplary immunofluorescence image showing D7 blastoid in close proximity to endothelial blood vessels. KRT7 indicates trophoblast, NANOG indicated epiblast and CD31 indicates vasculature.

FIGS. 23A-23C display non-limiting exemplary images Trophoblast-vascular functional interaction showing the cross-section of a day 7 blastoid cultured in the vascularized device indicates KRT7+ trophoblast cells are in close proximity to the CD31+ endothelial vessels. The media collected from the vascular and embryo compartment are both positive for hCG, an important marker of embryo implantation and trophoblast differentiation.

FIG. 24 depicts a non-limiting exemplary protocol for generating vascularized endometrial assembly.

FIG. 25 displays non-limiting exemplary results from gene expression analysis of stromal cells with hormonal stimulation.

FIG. 26 displays non-limiting exemplary results from gene expression analysis of endometrial epithelial organoids with hormonal stimulation.

FIGS. 27A-27B display non-limiting exemplary results from morphological analysis of endometrial epithelial organoids and stromal cells after hormonal priming.

FIGS. 28A-28C display non-limiting exemplary images of vascularized endometrial tissue.

FIG. 29 displays non-limiting exemplary images showing vasculature in the enclosed tissue area.

FIG. 30 displays non-limiting exemplary images showing the integration of endometrial tissue and vasculature.

FIG. 31 displays non-limiting exemplary images showing the integration of endometrial tissue and vasculature from Z stack analysis.

FIG. 32 displays non-limiting exemplary images showing the presence of luminal epithelial (LE), glandular epithelia (GE), endometrial stromal cells (ESCs) and endothelial blood vessels.

FIGS. 33A-33B display exemplary images showing an embryo implanted in a vascularized endometrium. The left panel of FIG. 33A shows a 9 dpf human embryo implanted in a vascularized endometrial tissue. The right panel of FIG. 33A shows AP2-Y+ trophoblast cells interacting with endometrial epithelial cells. The left panel of FIG. 33B endometrial epithelial cells differentiate into luminal epithelial (LE) and glandular epithelial cells (GE). The LE cells express SOX17 and endometrial stromal cells (ESC) are present in the area around the GE. The right panel of FIG. 33B shows trophoblast cells actively invading the tissue and interacting with LE and GE cells. Scale bar=100 μm.

FIGS. 34A-34C display exemplary images showing trophoblast cells migrating deep within the endometrial tissue. FIG. 34A shows a 9 dpf embryo implanted on the vascularized endometrial platform. AP2Y+ trophoblast cells interact with both luminal epithelial (LE) and glandular epithelial (GE) cells. FIG. 34B shows a zoomed view of an area of interest marked as “1” in FIG. 34A. Trophoblast cells are seen migrating away from the embryo. FIG. 34C shows a zoomed view of an area of interest marked as “2” in FIG. 34A. SOX17+ LE cells encapsulate the migratory trophoblast cells. Scale bar=100 μm.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include synthetic endometrial tissues, methods and uses thereof for assessing embryo implantation and growth. Upon implantation the embryo initiates growth, symmetry breaking, lineage specification and global reorganization to lay the foundations of the body plan. Concurrently, the trophectoderm cells in the blastocyst differentiate and invade the uterine vasculature to establish a nutrient delivery surface, the placenta, to facilitate transport of gases and nutrients from the mother to the fetus. These processes are vital for life, yet their underlying mechanisms are largely unknown because the implanted embryo is inaccessible within the mother. Uncovering the steps of trophectoderm invasion is of critical importance to understand and potentially overcome the bottlenecks for development in early life.

The events of implantation occur in a “black box” as the current understanding of its physico-chemical processes and their regulation is limited to correlative studies from human uterine lavage or biopsy samples or animal model. Myriad invasive treatment modalities have been suggested, and are indeed employed, for implantation failure. These include the assay of endometrial receptivity to give personalized timing of embryo transfer; intrauterine transfusion of platelet-rich plasma; and systemic immune therapies. Yet, evidence is lacking to inform whether there is truly any benefit from these treatments and new understanding and effective treatments are urgently needed.

To gain greater understanding of the requirements for successful implantation, the present disclosure provides an engineered bona fide feto-maternal interface comprised of a maternal uterus-like environment that nourishes the conceptus and facilitates correct development of embryos. This strategy provides an unparalleled resolution to monitor the primary steps of pregnancy and deciphers key determinants governing embryogenesis and placentation. The novel interface permits simultaneous access to both the developing embryo and its maternal surroundings, provides tools to assess the fundamental factors underlying gestational pathologies, and quantifies their implications.

Disclosed herein include a method of preparing a perfusable vascularized tissue for in vitro embryo implantation and growth. In some embodiments, the method comprises co-culturing endothelial cells and fibroblast cells in a hydrogel under a condition allowing the endothelial cells and fibroblast cells to self-organize into a perfusable vascularized tissue.

Disclosed herein includes a method of preparing a uterus-like tissue for in vitro embryo implantation and culturing. In some embodiments, the method comprises preparing a perfusable vascularized tissue according to the method described herein, priming endometrial epithelial organoids and stromal fibroblast cells with one or more hormones, and assembling the primed endometrial epithelial organoids and primed stromal fibroblast cells with the perfusable vasculature tissue to form a vascularized uterus-like tissue.

Disclosed herein also include a perfusable vascularized tissue and a uterus-like tissue for in vitro human embryo implantation and culturing obtainable from any one of the methods described herein.

Disclosed herein also includes a method of culturing a human embryo in vitro. In some embodiments, the method comprises preparing a perfusable vascularized tissue according to the method described herein and culturing a human embryo at pre-implantation stage in the perfusable vascularized tissue in a culture media under a condition allowing the human embryo to reach a post-implantation stage. In some embodiments, the method comprises preparing a uterus-like tissue structure according to any one of the method described herein, and culturing a human embryo at pre-implantation stage in the uterus-like tissue structure in a culture media under a condition allowing the human embryo to reach a post-implantation stage.

Disclosed herein also includes a microfluidic platform. In some embodiments, the microfluidic platform comprises a plurality of microfluidic devices each comprising at least one tissue chamber, wherein the at least one tissue chamber comprises an enclosed chamber, an exposed chamber accessible from a top plate via an opening, and interdigitated pillars at the interface between the enclosed chamber and the exposed chamber, the enclosed chamber in fluidic communication with the enclosed chamber, and wherein the enclosed chamber is fluidly connected to a media inlet and a media outlet, and the enclosed chamber and the exposed chamber each is connected to at least one hydrogel loading port.

As disclosed herein, an embryo can be a natural embryo or a stem-cell derived embryo. The embryo can be, e.g., a mammalian embryo, including a non-human embryo such as a mouse embryo, a rabbit embryo or a bovine embryo. As an example, the mammalian embryo can be a natural mammalian embryo or a stem-cell derived mammalian embryo. In some embodiments, the mammalian embryo is a human embryo (e.g., a natural human embryo or a stem-cell derived human embryo). In some embodiments, the mammalian embryo is a stem-cell derived embryo model (for example a blastoid). As can be appreciated by people of skill in the art, the platforms, methods, devices, systems and compositions disclosed herein can be used for blastoid models as well as the post-implantation models.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

As used herein, the term “differentiation” can refer to the process by which an unspecialized (“uncommitted”) or less specialized cell acquires the features of a specialized cell such as, for example, a neuronal cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell (which cells it came from and to what cells it can give rise). The lineage of a cell places the cell within a hereditary scheme of development and differentiation. As used herein, a “lineage-specific marker” can refer to a characteristic specifically associated with the phenotype of cells of a lineage of interest and can be used to assess the differentiation of an uncommitted cell to the lineage of interest.

The term “stem cell,” as used herein, refers to a cell that is capable of differentiating into one or more differentiated cell types. Stem cells may be totipotent. Stem cells may be pluripotent cells. Totipotent stem cells typically have the capacity to develop into any cell type. Totipotent stem cells are usually embryonic in origin. The term “progenitor cell,” as used herein, refers to a cell that is committed to a particular cell lineage and which gives rise to a particular limited range of differentiated cell types by a series of cell divisions. An example of a progenitor cell would be a myoblast, which is capable of differentiation to only one type of cell, but is itself not fully mature or fully differentiated.

As used herein, “markers”, “lineage markers” or, “lineage-specific markers” can refer to nucleic acid or polypeptide molecules that are differentially expressed in a cell of interest. Differential expression can mean an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art. In some embodiments, a marker can be enriched. The term “enriched”, as used herein, shall have its ordinary meaning, and can also refer to a statistically significant increase in levels of a gene product (e.g., mRNA and/or protein) in one condition as compared to another condition (e.g., in one cell layer as compared to another cell layer).

The term, “concentration” as used herein shall have its ordinary meaning, and can also refer to (a) mass concentration, molar concentration, volume concentration, mass fraction, molar fraction or volume fraction, or (b) a ratio of the mass or volume of one component in a mixture or solution to the mass or volume of another component in the mixture or solution (e.g., ng/ml). In some embodiments, the concentration can refer to fraction of activity units per volume (e.g., U/ml).

The term “analogue” as used herein refers to a compound which may be structurally related to the relevant molecule. The term “agonist” as used herein can refer to a compound which might not be structurally related to the relevant molecule. For example, an agonist may activate the relevant receptor by altering the conformation of the receptor. Nevertheless, in both cases the terms are used in this specification to refer to compounds or molecules which can mimic, reproduce or otherwise generally substitute for the specific biological activity of the relevant molecule.

As used herein the phrase “culture medium” or “media” refers to a liquid substance used to support the growth and development of stem cells and of an embryo. The culture medium used according to some embodiments of the invention can be a water-based medium which includes a combination of substances such as salts, nutrients, minerals, vitamins, amino acids, nucleic acids, and/or proteins such as cytokines, growth factors and hormones needed for cell growth and embryo development.

### Stem Cells and Embryo Development

Disclosed herein are synthetic vascularized, endometrial tissues and assemblies and related methods of generating the same. Disclosed herein also include methods of using the synthetic endometrial tissue structures for in vitro embryo implantation and culturing. In some embodiments, the synthetic vascularized tissue structures and methods disclosed herein can be used to culture a human embryo at a pre-implantation stage in a culture media under a condition allowing the human embryo to reach a post-implantation stage.

Mammalian embryogenesis is the process of cell division and cellular differentiation during early prenatal development which leads to the development of a mammalian embryo. While mammalian embryogenesis has some common features across all species, it will be appreciated that different mammalian species develop in different ways and at different rates. In general, though, the fertilized egg undergoes a number of cleavage steps (passing through two cell, four cell and eight cell stages) before undergoing compaction to form a solid ball of cells called a morula, in which the cells continue to divide. Ultimately the internal cells of the morula give rise to the inner cell mass and the outer cells to the trophectoderm. The morula in turn develops into the blastocyst, which is surrounded by trophectoderm and contains a fluid-filled vesicle, with the inner cell mass at one end.

The term “embryo” as used herein refers to a mammalian organism from the single cell stage. A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo.

In some embodiments, the embryo described herein is generated from culturing in vitro under appropriate conditions and resembles a natural embryo produced in vivo of a corresponding stage, such as having similar morphology, length, weight, cell type compositions and expression of developmental marker genes.

A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo. The earliest Carnegie stages are as follows in Table 1.

TABLE 1
Carnegie Stages of Development

| | Days since | |
|---|---|---|
| Carnegie | ovulation | |
| stage | (approx.) | Characteristic events/structures |
| 1 | 1 | fertilization; polar bodies |
| 2 | 2-3 | cleavage; morula; compaction |
| 3 | 4-5 | blastocyst and blastocoele; |
| | | trophoblast and embryoblast |
| 4 | 6 | syncytiotrophoblast; cytotrophoblast; |
| | | anchoring to endometrium |
| 5(a) | 7-8 | implantation; embryonic disc; bilaminar |
| | | germ disc; primary yolk sac; |
| 5(b) | 9-10 | formation of trophoblast lacunae; complete |
| | | penetration into endometrium; amniotic |
| | | cavity; primary umbilical vesicle |
| 5(c) | 11-16 | pre-chordal plate; extra-embryonic mesoblast; |
| | | secondary yolk sac |
| 6 | 17 | primitive streak, primitive node, primitive |
| | | groove; secondary umbilical vesicle; |
| | | primordial germ cells; body stalk; early |
| | | gastrulation. |
| 7 | 19 | Gastrulation; neural plate; start of |
| | | hematopoiesis. |
| 8 | 23 | Primitive pit |
| 9 | 25 | Neural groove; neural folds; septum |
| | | transversum; placode; early heart. |

In some embodiments, the mammalian embryos generated herein are mouse embryos. Theiler has established numbered stages of murine development. The earliest stages, as applied to (C57BLxCBA) F1 mice, arc described in the “emouse digital atlas” (www.emouseatlas.org) as follows in Table 2.

TABLE 2
Theiler Stages of Development

| Theiler | | Cell | |
|---|---|---|---|
| Stage | Dpc* (range) | number | (C57BL × CBA)Fl mice |
| 1 | 0-0.9 (0-2.5) | 1 | One-cell egg |
| 2 | 1 (1-2.5) | 2-4 | Dividing egg |
| 3 | 2 (1-3.5) | 4-16 (or 8-16) | Morula |
| 4 | 3 (2-4) | 16-40 (or 16-32) | Blastocyst, inner cell mass apparent |
| 5 | 4 (3-5.5) | | Blastocyst (zona-free) |
| 6 | 4.5 (4-5.5) | | Attachment of blastocyst; primary endoderm covers |
| | | | blastocoelic surface of inner cell mass |
| 7 | 5 (4.5-6) | | Implantation and formation of egg cylinder; |
| | | | Ectoplacental cone appears, enlarged epiblast, primary |
| | | | endoderm lines mural trophectoderm |
| 8 | 6 (5-6.5) | | Differentiation of egg cylinder. Implantation sites |
| | | | 2 × 3 mm. Ectoplacental cone region invaded by |
| | | | maternal blood, Reichert's membrane and proamniotic |
| | | | cavity form |
| 9a | 6.5 (6.25-7.25) | | Pre-streak(PS). advanced endometrial reaction, ecto |
| | | | lacental cone invaded by blood, extraembryonic |
| | | | ectoderm, embryonic axis visible |
| 9b | | | Early streak(ES), gastrulation starts, first evidence of |
| | | | mesoderm |
| 10a | 7 (6.5-7.75) | | Mid streak (MS), amniotic fold starts to form |
| 10b | | | Late streak, no bud (LSOB), exocoelom |
| 10c | | | Late streak, early bud (LSEB), allantoic bud first |
| | | | appears, node, amnion closing |
| 11a | 7.5 (7.25-8) | | Neural plate (NP), head process developing, amnion |
| | | | complete |
| 11b | | | Late neural plate (LNP), elongated allantoic bud |
| 11c | | | Early head fold (EHF) |
| 11d | | | Late head fold (LHF), foregut invagination |
| 12a | 8 (7.5-8.75) | | 1-4 somites, allantois extends, first branchial arch, |
| | | | heart starts to form, foregut pocket visible, preotic |
| | | | sulcus at 2-3 somite stage) |
| 12b | | | 5-7 somites, allantois contacts chorion at the end of |
| | | | TS12, Absent 2^{nd} arch, >7 somites |
| 13 | 8.5 (8-9.25) | | Turning of the embryo, 1^{st} branchial arch has |
| | | | maxillary and mandibular components, 2^{nd} arch |
| | | | present; Absent 3rd branchial arch |
| 14 | 9 (8.5-9.75) | | Formation & closure of ant. neuropore, otic pit |
| | | | indented but not closed, 3^{rd} branchial arch visible; |
| | | | Absent forelimb bud |
| 15 | 9.5 (9-10.5) | | Formation of post. neuropore, |
| | | | forelimb bud, forebrain vesicle subdivides; |
| | | | Absent hindlimb bud, Rathke's pouch |
| 16 | 10 (9.5-10.75) | | Posterior neuropore closes, Formation of hindlimb & |
| | | | tail buds, lens plate, Rathke's pouch; the indented nasal |
| | | | processes start |
| | | | to form; Absent thin & long tail |
| 17 | 10.5 (10-11.25) | | Deep lens indentation, adv. devel. of brain tube, tail |
| | | | elongates and thins, umbilical hernia starts to form; |
| | | | Absent nasal pits |
| 18 | 11 (10.5-11.25) | | Closure of lens vesicle, nasal pits, cervical somites no |
| | | | longer visible; |
| | | | Absent auditory hillocks, anterior footplate |
| 19 | 11.5 (11-12.25) | | Lens vesicle completely separated from the surface |
| | | | epithelium, Anterior, but no posterior, footplate. |
| | | | Auditory hillocks first visible; Absent retinal |
| | | | pigmentation and sign of fingers |
| 20 | 12 (11.5-13) | | Earliest sign of fingers, (splayedout), posterior |
| | | | footplate apparent, retina pigmentation apparent, |
| | | | tongue well-defined, brain vesicles clear; |
| | | | Absent 5 rows of whiskers, indented |
| 21 | 13 (12.5-14) | | Anterior footplate indented, elbow and wrist |
| | | | identifiable, 5 rows of whiskers, umbilical hernia now |
| | | | clearly apparent; |
| | | | Absent hair follicles, fingers separate distally |
| 22 | 14 (13.5-15) | | Fingers separate distally, only indentations between |
| | | | digits of the posterior footplate, long bones of limbs |
| | | | present, hair follicles in pectoral, pelvic and trunk |
| | | | regions; |
| | | | Absent open eyelids, hair follicles in cephalic region |
| 23 | 15 | | Fingers & Toes separate, hair follicles also in |
| | | | cephalic region but not at periphery of vibrissae, |
| | | | eyelids open; |
| | | | Absent nail primordia, fingers 2-5 parallel |
| 24 | 16 | | Reposition of umbilical hernia, eyelids closing, |
| | | | fingers 2-5 are parallel, nail primordia visible on |
| | | | Toes; |
| | | | Absent wrinkled skin, fingers & toes joined together |
| 25 | 17 | | Skin is wrinkled, eyelids are closed, umbilical hernia |
| | | | is gone; |
| | | | Absent ear extending over auditory meatus, long |
| | | | whiskers |
| 26 | 18 | | Long whiskers, eyes barely visible through closed |
| | | | eyelids, ear covers auditory meatus |
| 27 | 19 | | Newborn Mouse |

*“dpc” indicates days post conception, with the morning after the vaginal plug is found being designated 0.5 dpc or E0.5.

The platform, systems, methods, devices, and compositions described herein can be applied to embryos from any suitable mammalian species, such as: primates, including humans, great apes (e.g., gorillas, chimpanzees, orangutans), old world monkeys, new world monkeys; rodents (e.g., mice, rats, guinea pigs, hamsters); cats; dogs; lagomorphs (e.g., rabbits); cows; sheep; goats; horses; pigs; tigers; elephants; moose; deer; whales; fox; bears; lions; hippopotamuses; coyotes; seals; beavers; koalas; rhinoceros; dolphins; porcupines; sloth; platypus; chipmunks; hare; reindeer; cheetahs; hedgehogs; horses; bats; squirrels; kangaroos; echidna; springbok; opossums; marsupials; and any other livestock, agricultural, laboratory or domestic mammals. The mammals can be, for example, rodents, bats, eulipotyphlans (e.g., hedgehogs, moles and shrews), primates (e.g., humans, monkeys and lemurs), even-toed ungulates (e.g., pigs, camels, and whales), or carnivora (e.g., cats, dogs, and seals). The embryos can be, for example, natural or stem cell-derived non-human primate embryos, natural or stem cell-derived human embryos. The platform, systems, methods, devices and compositions described herein can be applied to embryos at various stages, e.g., pre-, peri-, and post-implantation stages. The platform, systems, methods, devices, and compositions described herein can be applied to an embryo from any non-human mammal, including but not limited to those described above. Thus, any of the culture media embodiments defined herein may be capable of supporting development of a non-human mammalian embryo on a substrate from a pre-implantation stage of development to a post-implantation stage of development.

In some embodiments, the mammalian embryos generated herein are human embryos. Human embryonic development is characterized by the processes of cell division and cellular differentiation of the embryo that occurs during the early stages of the development. A germinal stage of a human embryonic development refers to the time from fertilization through the development of the early embryo until implantation is completed in the uterus. The germinal stage takes about 10 days. During this stage, the one-celled zygote divides in a process referred to as cleavage. A blastocyst is then formed and implants in the uterus. Embryogenesis continues with the next stage of gastrulation, when the three germ layers of the embryo form in a process referred to as histogenesis, and the processes of neurulation and organogenesis follow.

The methods, synthetic materials, and culture media described herein can be used to assess the implantation and growth of a pre-implantation mammalian embryo. The term “pre-implantation stage” can be used herein to refer to a stage of development earlier than the stage corresponding to Theiler stage 7, Carnegie stage 5 (a), and corresponding stages in other species. As used herein, the term “post-implantation stage” can refer to a stage of development later than the stage corresponding to Theiler stage 7, Carnegie stage 5 (a), and corresponding stages in other species. A “post-implantation stage” may be determined by detecting the up-regulation of one or more genes by the embryo. For example, such a stage may be determined by detecting one or more of the following changes: the epiblast up-regulates Fgf5; the primitive endoderm differentiates into visceral endoderm that up-regulates Cer1 in a subpopulation of cells (the anterior visceral endoderm); the visceral endoderm up-regulates Eomes; and the trophectoderm up-regulate Hand1.

### Building Perfusable Synthetic Tissues for In Vitro Embryo Implantation and Growth

Provided herein include methods, compositions, and culture media for preparing synthetic perfusable tissue structures and three-dimensional models that mimic the structure and function of human endometrium. The synthetic tissues and three-dimensional models of human endometrium generated herein can be used for in vitro embryo implantation and culturing, providing valuable tools for investigating embryo implantation and feto-maternal interaction.

In some embodiments, methods and culture media for preparing a synthetic perfusable vascularized tissue for in vitro mammalian embryo (e.g., human embryo) implantation and culturing are disclosed. The synthetic perfusable vascularized tissue mimics a human vascularized endometrial tissue in vivo, therefore providing an in vitro environment for the implantation and growth of human embryos. In some embodiments, the method comprises co-culturing endothelial cells and fibroblast cells in a hydrogel under a condition allowing the endothelial cells and fibroblast cells to self-organize into a perfusable vascularized tissue. The term “perfuse”, “perfusable”, or “perfusion” used herein in connection with a synthesized tissue means a functional, lumenized vascular structure that allows for the continuous circulation and flow of media and delivery of oxygen and nutrients. The perfusable vascularized tissue generated herein can be used to culture both natural embryos and synthetic embryos generated in vitro from, for example, pluripotent stem cells such as embryonic stem cells.

Endothelial cells used herein are a specialized type of epithelial cell that form the inner lining of blood vessels and lymphatic vessels, and play a role in regulating blood flow, maintaining vascular permeability and mediating immune responses. The endothelial cells can comprise vascular endothelial cells, lymphatic endothelial cells, or both. Vascular endothelial cells line the entire circulatory system, from the heart to the smallest capillaries. Lymphatic endothelial cells line the lymphatic vessels, which are responsible for collecting excess fluid from tissues and returning it to the bloodstream. In some embodiments, the endothelial cells used herein are vascular endothelial cells. Exemplary vascular endothelial cells comprise continuous endothelium, fenestrated endothelium, discontinuous endothelium, and others identifiable by a person skilled in the art. Vascular endothelial cells can be organ specific in order to meet unique functional requirements. In some embodiments, the endothelial cells used herein are primary endothelial cells derived from umbilical cord such as human umbilical cord. Other primary endothelial cells are also suitable for the methods described herein. Exemplary endothelial cells suitable for the methods herein include, but are not limited to, endothelial progenitor cells derived from umbilical cord blood, mouse brain endothelial cells, and immortalized vascular endothelial cell lines such as EA.hy926, HMEC-1, TIME cells, CI-huMEC, EC-RF24, iMAEC and others as will be understood by a person skilled in the art. In some embodiments, the endothelial cells used herein are human umbilical cord vascular endothelial cells (HUCVE cells).

Fibroblast cells used herein are a type of connective tissue cell that plays a crucial role in maintaining the structure and function of tissues, and are responsible for producing and maintaining the extracellular matrix, a network of proteins and other molecules that provides support and structure to tissues. The primary types of fibroblasts include, for example, pericytes, cardiac fibroblasts, muscular fibroblasts, dermal fibroblasts, fat fibroblasts, and other types associated with the structure and function of specific organs such as the colon, bladder, lungs, and digestive organs. The fibroblast cells used herein can comprise uterine fibroblasts, intestinal fibroblasts, skin fibroblasts, liver fibroblasts, lung fibroblasts, kidney fibroblasts, cardiac fibroblasts, bladder fibroblasts, or any combination thereof. In some embodiments, the fibroblast cells used herein are fibroblasts derived from a mammalian such as a human. In some embodiments, the fibroblast cells used herein are human uterine fibroblast cells, human primary brain vascular fibroblast, mouse embryonic brain vascular fibroblast, human dermal fibroblast, human perivascular fibroblast, human lung fibroblast, or any combination thereof. In some embodiments, the fibroblast cells used herein are human lung fibroblast cells.

The endothelial cells and fibroblast cells are co-cultured in a hydrogel under a condition allowing the endothelial cells and fibroblast cells to self-organize into a perfusable vascularized tissue. Other cells (e.g., endometrial cells) can, for example, be co-cultured in the hydrogel with the endothelia cells and/or fibroblast cells as well. For example, in some embodiments, the endometrial cells, the endothelial cells and fibroblast cells can be co-cultured in a hydrogel under a condition allowing the cells to self-organize into a perfusable vascularized tissue. In some embodiments, trophoblast cells can be cultured in any of the methods, platforms, compositions, systems and devices described herein. The term “hydrogel,” as used herein, shall be given its ordinary meaning, and shall also refer to a gel in which water is the dispersion medium. Typically, a hydrogel comprises a plurality of polymer molecules that are cross-linked, either via covalent bonds or via non-covalent interactions, thus forming a polymer scaffold, also referred to herein as a hydrogel scaffold. A hydrogel can refer to a water-swellable polymeric matrix formed from a three-dimensional network of macromolecules held together by covalent or non-covalent crosslinks, that can absorb a substantial amount of water (by weight) to form a gel. A hydrogel can refer to a network of polymer chains in an aqueous dispersion medium. In some preferred embodiments, the cross-linking is via covalent bonds. Cross-linking typically comprises inter-polymer bonds (bonds between different polymer molecules), but may also comprise intra-polymer bonds (bonds within the same polymer molecule). In some embodiments, the polymers are water-soluble in their non-cross-linked form, but are insoluble once they are cross-linked.

The hydrogels provided herein can comprise hydrogel scaffolds made of cross-linked polymers. The term “cross-linked,” as used herein, can refer to a type of binding involving a plurality of polymers and a plurality of binding interactions. Cross-linked polymers are polymers that are connected to form a network, and, in the context of hydrogels, a hydrogel scaffold. Accordingly, a polymer in a cross-linked state is connected to another polymer or a plurality of other polymers through two or more covalent bonds or non-covalent interactions, thus forming a network of interconnected polymer molecules. Cross-linking can be either via covalent bonds or via non-covalent interactions. In some embodiments, hydrogel-forming polysaccharides cross-link via non-covalent bonds, e.g., as is the case for alginates, via chelation of ions. In other embodiments, however, the polymers forming the hydrogel scaffold of a hydrogel provided herein are cross-linked via covalent bonds. The formation of such covalently cross-linked hydrogel scaffolds can involve the formation of covalent bonds between individual polymer molecules, but may also involve the formation of intra-molecular bonds within the same polymer molecule.

The plurality of polymers can be cross-linked. For example, the plurality of polymers can be cross-linked via (1) covalent bonds and/or non-covalent bonds, (2) inter-polymer bonds and/or intra-polymer bonds, and/or (3) physical crosslinks and/or chemical crosslinks. The physical crosslinks can be formed by a mechanism selected from the group consisting of ionic interactions, hydrophobic interactions, hydrogen bonding, Van der Waals forces and desolvation. The chemical crosslinks can be formed by a mechanism selected from the group consisting of free radical polymerization, condensation polymerization, anionic or cationic polymerization and step growth polymerization. In some embodiments, the plurality of payload molecules and/or the plurality of gas vesicles are not covalently bound to the polymer scaffold.

The term “polymer,” as used herein, can refer to a molecule comprising a plurality of repeating structural units (monomers), typically at least 3, linked together via covalent bonds. Non-limiting examples of polymers are polysaccharides, polynucleotides, and polypeptides. Exemplary hydrogel-forming polymers, e.g., dextrose, carboxymethylcellulose, and hyaluronic acid, are described in more detail elsewhere herein. Additional polymers that can form hydrogels are also encompassed. It will be apparent to the skilled artisan that any suitable hydrogel can be employed in some embodiments of this disclosure, and that the exemplary hydrogel-forming polymers described herein in more detail are not in any way limiting. For example, in some embodiments, hydrogels are provided that comprise polymers that are known in the art to be useful in the preparation of hydrogels. Such polymers may include, in some embodiments, e.g., cellulose derivatives, xyloglucans, chitosans, glycerophosphates, alginates, gelatin, polyethylene glycol, N-isopropylamide copolymers (e.g., poly(N-isopropylacrylamide-co-acrylic acid) or poly(N-isopropylacrylamide)/poly(ethylene oxide)), poloxamers (e.g., pluronic-modified poloxamer or poloxamer/poly(acrylicacid)), poly(ethylene oxide)/poly(D,L-lactic acid-co-glycolic acid), poly(organophosphazene), or poly(1,2-propylene phosphate), and their derivatives. Additional polymers useful for the formation of a hydrogel in the context of some embodiments of this disclosure will be apparent to those of skill in the art, and the disclosure is not limited in this respect.

The plurality of polymers can comprise a natural polymer (e.g., alginate, agarose, carrageenan, chitosan, dextran, carboxymethylcellulose, heparin, hyaluronic acid, polyamino acid, collagen, gelatin, fibrin, a fibrous protein-based biopolymer, or any combination thereof). A “natural polymer” can refer to a polymeric material that may be found in nature. In various embodiments, hydrogels are formed by natural polymers selected from the group consisting of polysaccharides, glycosaminoglycans, proteins, and mixtures thereof. These hydrogels may also be termed herein as “natural hydrogels”. Polysaccharides are carbohydrates which may be hydrolyzed to two or more monosaccharide molecules. They may contain a backbone of repeating carbohydrate (sugar unit). Examples of polysaccharides include, but are not limited to, alginate, agarose, chitosan, dextran, starch, and gellan gum. Glycosaminoglycans are polysaccharides containing amino sugars as a component. Examples of glycosaminoglycans include, but are not limited to, hyaluronic acid, chondroitin sulfate, dermatin sulfate, keratin sulfate, dextran sulfate, heparin sulfate, heparin, glucuronic acid, iduronic acid, galactose, galactosamine, and glucosamine.

Examples of natural hydrogels which are well known in the art include alginate and agarose. In some embodiments, the degradable hydrogel comprises alginate. The term “alginate” can refer to any of the conventional salts of algin, which is a polysaccharide of marine algae, and which may be polymerized to form a matrix for use in drug delivery and in tissue engineering due to its biocompatibility, low toxicity, relatively low cost, and simple gelation with divalent cations such as calcium ions (Ca2+) and magnesium ions (Mg2+). Examples of alginate include sodium alginate which is water soluble, and calcium alginate which is insoluble in water. In some embodiments, agarose may be used as the hydrogel. Agarose can refer to a neutral gelling fraction of a polysaccharide complex extracted from the agarocytes of algae such as a Rhodophyceae. However, unlike alginate, it forms thermally reversible gels.

The plurality of polymers can comprise a synthetic polymer, such as, for example, alginic acid-polyethylene glycol copolymer, poly(ethylene glycol), poly(2-methyl-2-oxazoline), poly(ethylene oxide), poly(vinyl alcohol), poly(acrylamide), poly(n-butyl acrylate), poly-(a-esters), poly(glycolic acid), poly(lactic-co-glycolic acid), poly(L-lactic acid), poly(N-isopropylacrylamide), butyryl-trihexyl-citrate, di(2-ethylhexyl) phthalate, di-iso-nonyl-1,2-cyclohexanedicarboxylate, expanded polytetrafluoroethylene, ethylene vinyl alcohol copolymer, poly(hexamethylene diisocyanate), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), poly(ethylene), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), or any combination thereof.

The plurality of polymers can comprise a polymer selected from the group comprising collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethylene oxide)-poly(propylene oxide)-poly(ethylene oxide) copolymer, copolymers thereof, or any combination thereof.

The plurality of polymers can comprise polystyrene, neoprene, polyetherether 10 ketone (PEEK), carbon reinforced PEEK, polyphenylene, PEKK, PAEK, polyphenylsulphone, polysulphone, PET, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, TEFLON® TFE, polyethylene terephthalate (PETE), TEFLON® FEP, TEFLON® PFA, and/or polymethylpentene (PMP) styrene maleic anhydride, styrene maleic acid, polyurethane, silicone, polymethyl methacrylate, polyacrylonitrile, poly(carbonate-urethane), poly(vinylacetate), nitrocellulose, cellulose acetate, urethane, urethane/carbonate, polylactic acid, polyacrylamide (PAAM), poly(N-isopropylacrylamine) (PNIPAM), poly(vinylmethylether), poly(ethylene oxide), poly(ethyl(hydroxyethyl) cellulose), poly(2-ethyl oxazoline), polylactide (PLA), polyglycolide (PGA), poly(lactide-co-glycolide) PLGA, poly(e-caprolactone), polydiaoxanone, polyanhydride, trimethylene carbonate, poly(β-ydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethylene glycol (PEG), polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-i sopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, ethylene vinyl acetate, polyethylene oxide, PEG or any of its derivatives, PLLA, PDMS, PIPA, PEVA, PILA, PEG styrene, Teflon RFE, FLPE, Teflon FEP, methyl palmitate, NIPA, polycarbonate, polyethersulfone, polycaprolactone, polymethyl methacrylate, polyisobutylene, nitrocellulose, medical grade silicone, cellulose acetate, cellulose acetate butyrate, polyacrylonitrile, PLCL, and/or chitosan.

In various embodiments, hydrogels provided herein are selected from the group consisting of hydrogels made from a hydrophilic monomer, hydrogels made from a hydrophilic polymer, hydrogels made from a hydrophilic copolymer, and combinations thereof. In some embodiments, hydrogels provided herein are made from hydrophilic polypeptides.

The hydrogel composition can comprise a water content between about 10% and about 100% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values).

The polymer scaffold can comprise about 0.000000001%, to about 90% (e.g., 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) volume per volume (% v/v), weight per volume (% w/v) or weight per weight (% w/w) of the hydrogel composition.

The hydrogel used herein can be a natural hydrogel or a synthetic hydrogel. In some embodiments, the hydrogel comprises natural hydrogels such as collagen or hyaluronic acid or synthetic hydrogels such as PEG or polyacrylamide. In some embodiments, the hydrogel used herein is made from polypeptides. Hydrogel can be prepared, for example, by warming of a collagen type-I solution to promote polymerization, by addition of biological accelerants such as thrombin to fibrinogen, or by photo cross-linking of polymers.

In some embodiments, the hydrogel used herein is fibrin hydrogel. Fibrin is typically composed of fibrinogen and thrombin at a certain ratio suitable for forming a hydrogel structure. In some embodiments, the fibrin used herein comprises fibrinogen cross-linked using thrombin at a thrombin: fibrinogen ratio of about 1:2, 1:3, 1:4, 1:5, 1:6, 1:7, or a number or a range between any two of these values. In an exemplary embodiments, thrombin and fibrinogen are provided a ratio of about 1:5.

In some embodiments, the hydrogel described herein is functionalized with one or more extracellular matrix component such as extracellular matrix proteins to improve cell adhesion and growth of vasculature. The extracellular matrix components are a diverse group of molecules that form a complex network outside of cells, providing structural support and regulating cell behavior. Exemplary extracellular matrix components include collagens, elastin, laminins, fibronectins, proteoglycans, tenascins, glycoproteins, hyaluronic acid, and others identifiable to a person skilled in the art. In some embodiments, the hydrogel is functionalized with ECM components such as laminin, fibronectin, collagen (e.g., collagen I-V, optionally collagen I and collagen IV), hyaluronic acid, or any combination thereof. The degree of functionalization can vary in different embodiments. In some embodiments, the degree of functionalization can vary from about 5% to about 60% (e.g., 5%, 10%, 15%, 20%, 25%, 30%, 35%, 40%, 45%, 50%, 55%, 60%, or a number or a range between any two of these values). In some exemplary embodiments, the hydrogel used herein are fibrin functionalized with about 10%-30% laminin and about 10%-30% fibronectin. In an exemplary embodiments, the hydrogel used herein are fibrin comprising thrombin and fibrinogen provided a ratio of about 1:5 and functionalized with about 20% laminin and about 20% fibronectin. In another exemplary embodiments, the hydrogel used herein comprise about 60% fibrin, 20% collagen I, 10% laminin, and 10% fibronectin. The thrombin and fibrinogen are provided in the fibrin at a ratio of about 1:5.

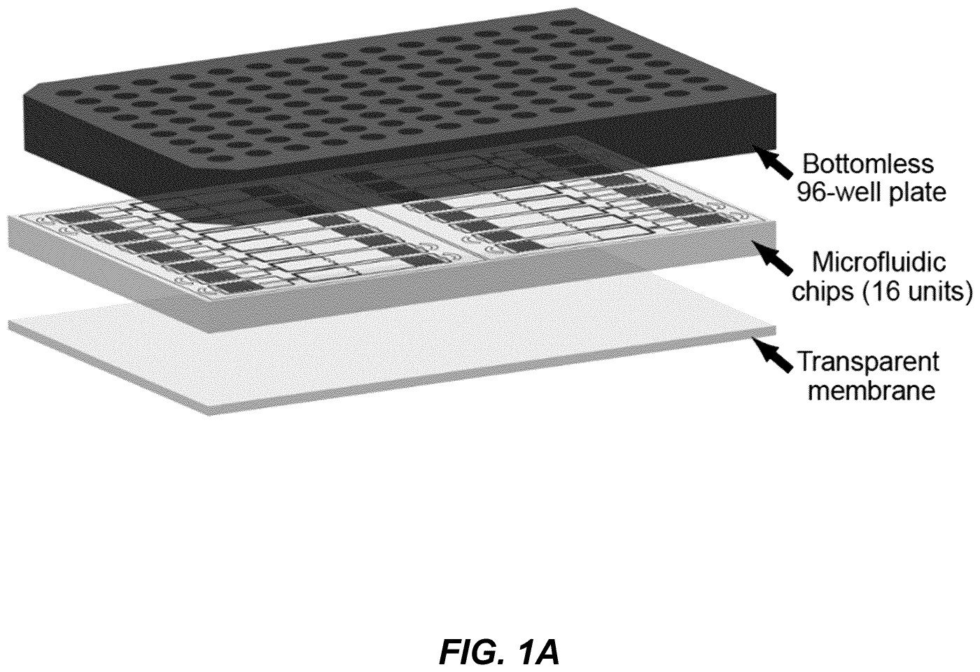

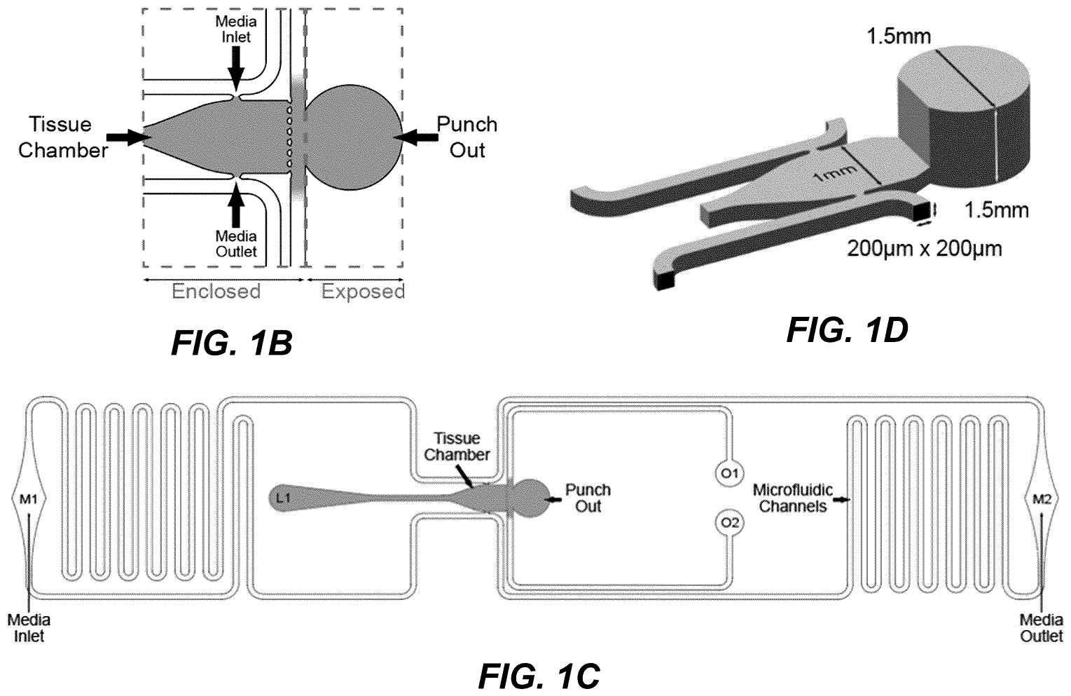

In some embodiments, co-culturing endothelial cells and fibroblast cells in a hydrogel takes place in a microfluidic device. A microfluidic device suitable for generating a perfusable vascularized tissue can comprise a microfluidic chip containing a plurality of microfluidic implantation devices (see, for example, FIG. 1A). Each microfluidic implantation device can comprise a tissue chamber (see, for example, FIGS. 1B-C), wherein endothelial cells and fibroblast cells can be loaded with a suitable hydrogel described herein. Each tissue chamber can comprise two main chambers: an enclosed chamber and an exposed chamber. The enclosed chamber is a microfluidic channel that is enclosed within a base plate and not accessible from the top plate, while the exposed chamber is open to the top of the plate via a circular opening termed as a “punch out” providing access to the chamber from the top. Both the enclosed and exposed chambers are part of microfluidic channels and in fluidic communication with one another. The tissue chamber further comprises inter-digitated line of pillars (FIGS. 1B and 1D) at the interface between the enclosed chamber and the exposed chamber. Therefore, when endothelial cells, fibroblast cells and hydrogels are seeded in the enclosed chamber, they can flow into the exposed chamber and partially fill it. The tissue chamber, particularly the enclosed chamber, are connected to a media inlet and a media outlet (M1 and M2) that run along the device (FIGS. 1B-C). Additionally, the enclosed chamber and the exposed chamber each can be connected to at least one port. For example, the enclosed chamber can be connected to L1 shown in FIG. 1C and the exposed chamber can be connected to O1 and O2. In some embodiments, the port(s) connected to the enclosed chamber and the exposed chamber are different. This setup allows for the growth of a continuous and closed-loop vascular network in the hydrogel encompassing the two chambers. In some embodiments, ports such as L1, O1 and O2 are designed for loading the cell-hydrogel solution, while the media inlet/outlet are for supplying media such as the vasculature media. In some embodiments, the microfluidic chip is placed under a top plate (FIG. 1A), and the ports and chambers can be accessed through the wells in the top place. In some embodiments, each implantation device unit occupies 6 wells in the top plate, and there are a total of 16 units lined up in the microfluidic chip, which occupies all 96 wells of the plate.

In some embodiments, co-culturing endothelial cells and fibroblast cells in a hydrogel comprises mixing endothelial cells and fibroblast cells with a hydrogel and then seeding the mixture in the enclosed chamber via one or more ports (e.g., L1 port in FIG. 1C) connected to the enclosed chamber. The endothelial cells and fibroblast cells can be seeded at a cell density suitable for the cells to grow and assemble in the hydrogel. In some embodiments, the endothelial cells and fibroblast cells are seeded in the enclosed chamber at a density of about 2-4 M cells/ml (optionally, 3 M cells/ml) each cell type into the enclosed chamber via a port. The method can further comprise seeding endothelial cells, fibroblast cells and hydrogels in the exposed chamber via one or more ports connected to the exposed chamber (e.g., O1 and/or O2 in FIG. 1C). In some embodiments, the port(s) connected to the exposed chamber can be different from the port(s) connected to the enclosed chamber. This can create a continuous cell-hydrogel solution in both the enclosed and exposed chamber. The cell density of the endothelial cells and fibroblast cells seeded in the exposed chamber can be the same as or different from the cell density in the enclosed chamber. The volume of the cell-hydrogel mixture in the enclosed chamber and in the exposed chamber can be the same or different. In some embodiments, the seeding volume in the exposed chamber can be higher than the seeding volume in the enclosed chamber to achieve a taller vascular bed in the exposed chamber.

Once the hydrogel mixture is solidified, a laminin solution and/or a vasculature media can be added to the chambers, e.g., via the media inlet and/or media outlet (M1 and/or M2 in FIG. 1C). Accordingly, the method can further comprise adding a laminin solution to a solidified hydrogel mixture comprising the endothelial cells and fibroblast cells and incubating for a time period (e.g., 5, 10, 15, 20, 25 minutes). The laminin solution can then be replaced with a vasculature media suitable for growing endothelial cells such as an endothelial cell growth medium (EGM). A hydrostatic gradient of the vasculature media can be maintained in each device between the media inlet and the media outlet to generate a media flow from a higher pressure area to a lower pressure area.

A vasculature media used herein typically is a culture media suitable for endothelial cell growth containing a basal medium supplemented with various growth factors, hormones, and other nutrients to create a suitable environment for growing endothelial cells. Exemplary commercial endothelial cell growth media include, for example, endothelial cell growth medium-2 (EGM-2) and VascuLife EnGS media among others identifiable by a person skilled in the art.

In some embodiments, the vasculature media comprises a basal culture medium providing essential salts, amino acids, vitamins, and carbohydrates. In some embodiments, the basal culture medium comprises Dulbecco's Modified Eagle Media (DMEM), DMEM Nutrient Mixture 12 (DMEM/F12), a non-human serum or serum substitute thereof, a reducing agent, an antibiotic or an antimicrobial, L-glutamine or an analogue thereof, or any combination thereof. In some embodiments, the non-human serum or serum substitute comprises fetal bovine serum, bovine serum albumin, KnockOut™ Serum Replacement, or any combination thereof. The reducing agent can comprise, for example, beta-mercaptoethanol (BME), N-acetyl-L-cysteine, dithiothreitol (DTT), or any combination thereof. In some embodiments, the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof.

The vasculature media can further comprise one or more growth factors that promote cell proliferation and differentiation. For example, the vasculature media can comprise human fibroblast growth factor-B (hFGF-B) for stimulating endothelial proliferation, vascular endothelial growth factor (VEGF) for promoting angiogenesis, R3-insulin-like growth factor-1 (R3-IGF-1) for enhancing growth, and human epidermal growth factor (hEGF) for stimulating proliferation. In some embodiments, the vasculature media used herein comprises ascorbic acid (vitamin C) as an antioxidant and collagen synthesis support. In some embodiments, the vasculature media used herein comprises an anticoagulant, optionally heparin. In some embodiments, the vasculature media used herein comprises hydrocortisone for modulating inflammation and promotes cell survival.

In some embodiments, the EGM medium comprises fetal bovine serum, hydrocortisone, human fibroblast growth factor-B (hFGF-B), vascular endothelial growth factor (VEGF), R3-insulin-like growth factor-1 (R3-IGF-1), ascorbic acid, heparin, human epidermal growth factor (hEGF), an antimicrobial, or any combination thereof. In some embodiments, the fetal bovine serum is provided at about 2% final concentration.

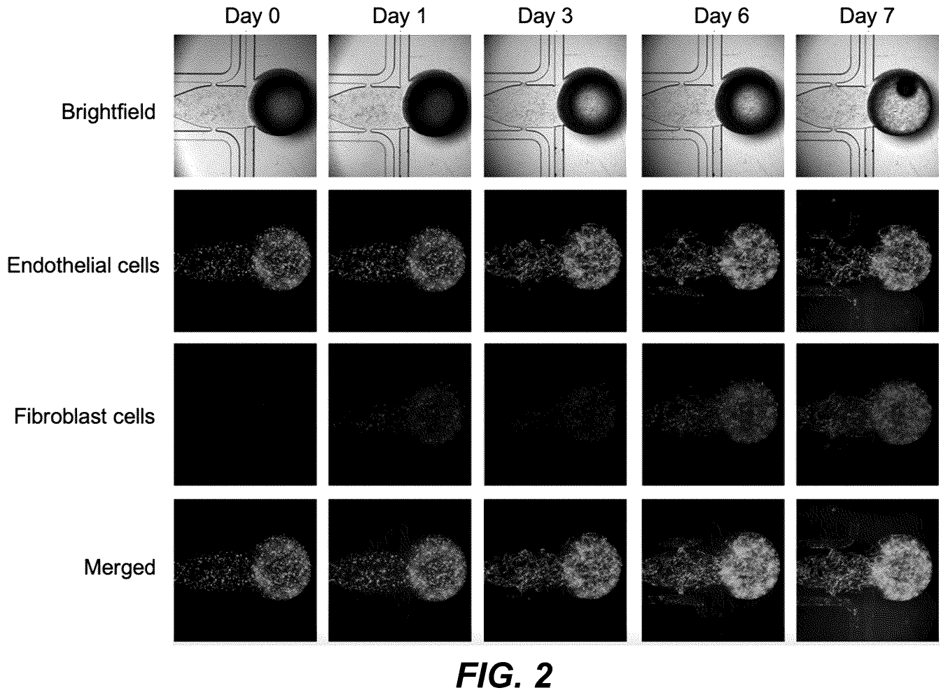

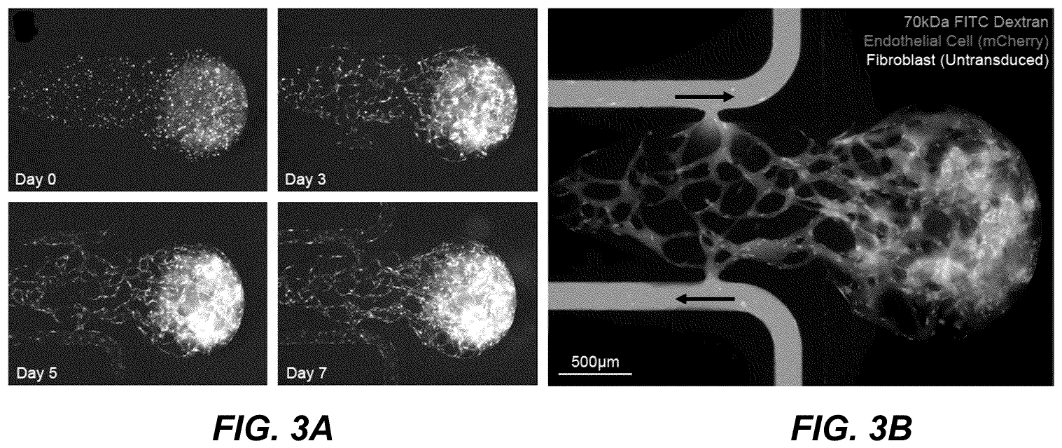

In some embodiments, the endothelial cells and fibroblast cells can be cultured in a vasculature media for at least 5-6 days (e.g., 5, 6, 7, 8, or more days) allowing the endothelial cells and fibroblast cells to self-organize into a closed-loop and continuous vasculature in the hydrogel. The co-culturing allows the vasculature to fuse with microfluidic channels to create a perfusable network. In some embodiments, the fibroblast cells can acquire a pericyte-like morphology and help stabilize vascular tubules (see, for example, FIG. 2). In some embodiments, the vasculature in the enclosed chamber anastomoses into the media inlet/outlet channels (see, for example, FIG. 3A), which allows the perfusion of media from the higher-pressure side to the lower-pressure side via the vasculature.

Provided herein also include methods and compositions for preparing a uterus-like assembly that mimics the in vivo environment of a natural uterus such as a human uterus. The uterus-like model generated herein can be used for in vitro mammalian embryo (e.g., human embryo) implantation and culturing, as well as for investigating endometrial development, diseases, and early stages of pregnancy.

In some embodiments, the method of preparing a uterus-like assembly comprises preparing a perfusable vascularized tissue using the methods described above, priming endometrial epithelial organoids and stromal cells with one or more hormones, and assembling the primed endometrial epithelial organoids and primed stromal cells with the perfusable vascularized tissue to form a uterus-like assembly structure.

Endometrial epithelial organoids are three-dimensional cell cultures that mimic the structure and function of the endometrial lining of the uterus in vitro. Endometrial epithelial organoids can be derived from endometrial tissue and can be maintained long-term, retaining key features of the tissue of origin, including hormone responsiveness and cell composition. Endometrial epithelial organoids are typically generated through a process involving the isolation of epithelial cells from endometrial tissue and their subsequent embedding in a suitable extracellular matrix and culturing in a suitable medium as would be understood by a person skilled in the art. Stromal cells are a type of cells found within a variety of organs and tissues including bone marrow, connective tissue of various organs, organs, and are crucial for providing structural support, maintaining tissue integrity, and influencing the behavior of other cells within the organ. These cells are heterogeneous and can include fibroblasts, pericytes, mesenchymal stem cells (MSCs), and endothelial cells, among others.

The endometrial epithelial organoids and stromal cells are primed with one or more hormones including estrogen, an estrogen analogue, or an estrogen receptor agonist, and progesterone, a progesterone analogue, or a progesterone receptor agonist. In some embodiments, endometrial epithelial organoids and stromal cells are primed with B-estradiol, medroxyprogesterone acetate, progesterone, cyclic adenosine monophosphate, or any combination thereof. In some embodiments, the endometrial epithelial organoids and stromal cells are primed separately, and then assembled with a pre-made vascularized tissue. This ensures proper growth and transformation of vasculature and endometrial cells prior to assembly as they require different media and reagents.

In some embodiments described herein, trophoblast organoids can be substituted for endometrial organoids or combined with endometrial organoids and stromal cells to generate endometrial-trophoblast assembloids. Also, the co-culturing can incorporate additional cells, e.g., immune cells and endothelial cells, in accord with the teachings herein.

Following the assembly of the endometrial epithelial organoids and stromal cells with the pre-made vascularized tissue, the assembloid is then supplied with a minimum differentiation media, with a continued treatment of the hormone(s). Accordingly, in some embodiments, the assembling comprises culturing the primed endometrial epithelial organoids and primed stromal cells in the perfusable vascularized tissue in a minimal differentiation media in the presence of the one or more hormones (e.g., B-estradiol, medroxyprogesterone acetate, progesterone, and/or cyclic adenosine monophosphate). The minimal differentiation media can comprise a basal medium supplemented with N-acetylcysteine (NAC), Oestradiol (E2), a cyclic AMP analogue (e.g., 8-bromo-cAMP), and progestin (e.g., medroxyprogesterone acetate (MPA)). Oestradiol (E2) promotes proliferation of gland-like organoids and cooperates with NOTCH signalling to activate ciliogenesis in a subpopulation of EpC (Haider et al., 2019). Treatment with a progestin (e.g. MPA) and a cyclic AMP analogue (e.g. 8-bromo-cAMP) can induce secretory transformation of gland-like organoids in parallel with expression of luteal-phase marker genes (Turco et al., 2017; Boretto et al., 2017).

To ensure the compatibility of the cell types in the assembled structure, in some embodiments, a hydrogel composition used herein comprises about 60% fibrin (8 mg/mL), about 20% collagen-I (5 mg/mL), about 10% laminin, and about 10% fibronectin. In some embodiments, the fibrin used herein comprises fibrinogen cross-linked using thrombin at a thrombin: fibrinogen ratio of about 1:2, 1:3, 1:4, 1:5, 1:6, 1:7, or a number or a range between any two of these values. In an exemplary embodiments, thrombin and fibrinogen are provided a ratio of about 1:5.

In some embodiments, assembling the primed endometrial epithelial organoids and primed stromal cells with the perfusable vascularized tissue to form a uterus-like tissue takes place in a microfluidic device described herein. The method can further comprise loading the primed endometrial epithelial organoids and primed stromal cells into a tissue chamber of the microfluidic device, for example, through an opening (punch out) in the exposed chamber which is accessible from the top of the plate, wherein the tissue chamber contains a pre-made perfusable vascularized tissue. The minimum differentiation media as well as the hormones can be supplied to the tissue chamber via the media inlet/outlet.

Provided herein also includes a perfusable vascularized tissue for in vitro mammalian embryo implantation and culturing. The perfusable vascularized tissue can be obtained using the method described herein. Provided herein also includes a uterus-like tissue for in vitro mammalian embryo implantation and culturing. The uterus-like tissue can be obtained using the method described herein. The uterus-like tissue generated herein exhibits luminal epithelial cells, glandular epithelial cells, stromal cells, fibroblast and endothelial vessels similar to a natural uterus.

### Implantation and Growth of Embryos

Provided herein include methods, compositions, and culture media for implanting and growing mammalian embryos or embryo s in vitro using the synthetic tissue structures generated herein, including the perfusable vascularized tissue and uterus-like structure. The mammalian embryos can be human embryos or non-human embryos. In some embodiments, the embryo models are stem-cell derived embryo models such as blastoids or post-implantation models such as embryoids.

In some embodiments, the method comprises preparing a perfusable vascularized tissue according to the method described herein and culturing a mammalian embryo at pre-implantation stage in the perfusable vascularized tissue in a culture media under a condition allowing the mammalian embryo to reach a post-implantation stage. In some embodiments, the method comprises preparing a uterus-like tissue structure using the method described herein, and culturing a human embryo at pre-implantation stage in the uterus-like tissue in a culture media under a condition allowing the human embryo to reach a post-implantation stage. In some embodiments, the mammalian embryo is at day 5 after fertilization and cultured in the perfusable vascularized tissue or the uterus-like tissue structure until it reaches at least Carnegie stage 5c or beyond. In some embodiments, the mammalian embryo is a human embryo.

In some embodiments, the embryo subject to culturing in the perfusable vascularized tissue or in the uterus-like tissue structure is at day 5 after fertilization or at Carnegie stage 3-2 prior to culturing in the perfusable vascularized tissue or the uterus-like tissue structure. In some embodiments, the embryo subject to culturing in the perfusable vascularized tissue or in the uterus-like tissue structure is at a blastocyst stage. In some embodiments, the embryo is cultured in the perfusable vasculature tissue or in the uterus-like tissue structure for at least 6 days (e.g., 6, 7, 8, 9, 10 or more days) until the embryo reaches day 12 after fertilization or beyond. In some embodiments, the embryo (e.g., human embryo) is cultured for a time period until the embryo reaches at least Carnegie stage 5c or beyond. Prior to the culturing, the perfusable vascularized tissue can be supplied with a vasculature media suitable for endothelial cell growth as described above. For example, the vasculature media can comprise fetal bovine serum, hydrocortisone, human fibroblast growth factor-B (hFGF-B), vascular endothelial growth factor (VEGF), R3-insulin-like growth factor-1 (R3-IGF-1), ascorbic acid, heparin, human epidermal growth factor (hEGF), an antimicrobial, or any combination thereof. The uterus-like tissue structure can be supplied with a minimum differentiation media as described above. For example, a minimum differentiation media can comprise a basal culture media supplemented with N-acetylcysteine, estradiol, a cyclic AMP analogue, optionally, 8-bromo-cAMP, and a progestin, optionally, medroxyprogesterone acetate (MPA). In some embodiments, during the culturing of the embryo, neither the vasculature media nor the minimum differentiation media is supplied to the tissue structures.

In some embodiments, culturing the mammalian embryo (e.g., human embryo) in the perfusable vascularized tissue or the uterus-like tissue structure comprises transferring the human embryo to the perfusable vasculature tissue or the uterus-like tissue structure. The embryo can be placed on the top of the perfusable vascularized tissue or the uterus-like tissue structure or embedded in the tissues structure. In some embodiments, transferring the embryo to the perfusable vasculature tissue or the uterus-like tissue structure is performed by placing the embryo on the top of the perfusable vascularized tissue or the uterus-like tissue structure. In some embodiments, the method comprises transferring the embryo to the perfusable vascularized tissue (e.g., placing the embryo on the top of the perfusable vascularized tissue) or the uterus-like tissue structure and embedding the embryo in the perfusable vascularized tissue or the uterus-like tissue structure. Transferring and embedding the embryo can occur concurrently or sequentially. For example, transferring the embryo to the perfusable vascularized tissue is followed by embedding the embryo in the tissue. In some embodiments, transferring the embryo and embedding the embryo can be separated by a time interval of at least one day. In some embodiments, transferring the embryo and embedding the embryo is separated by a time interval of two days. In some embodiments, transferring a human embryo to the perfusable vascularized tissue or the uterus-like tissue structure occurs at day 6 after fertilization and embedding the human embryo in the perfusable vascularized tissue or the uterus-like tissue structure occurs two days after (at day 8 after fertilization). In some embodiments, transferring the embryo and embedding the embryo occur concurrently or on the same day. For example, transferring a human embryo to the perfusable vascularized tissue or the uterus-like tissue structure and embedding the human embryo in the perfusable vascularized tissue or the uterus-like tissue structure both occur at day 6 after fertilization.

In some embodiments, the human embryo is cultured under a dynamic condition comprising a progressive increase of oxygen concentration, gas pressure, and/or serum concentration in the culture media. In order to modulate the oxygen tension and/or gas pressure in the culture media, the perfusable vascularized tissue or the uterus-like tissue structure containing the embryo can be placed in an incubator or a culture chamber. The oxygen concentration can be modulated by supplying oxygen at an increasing concentration to the culture chamber. In some embodiments, the culture chamber can have an atmosphere comprising an increasing oxygen concentration from about 5% to about 25% oxygen level or higher, optionally from about 5% to about 12%. The oxygen concentrations can be increased throughout the culturing starting from about 5%, to 6%, 7%, 8%, 9%, 10%, 11%, 12%, 13%, 14%, 15%, 16%, 17%, 18%, 19%, 20%, 21%, 22%, 23%, 24%, 25% or higher at any suitable time interval (e.g., daily interval), optionally from about 5%, to 6%, 7%, 8%, 9%, 10%, 11%, or 12% (e.g., daily interval). In some embodiments, increasing of oxygen concentration is effected every 0.5-2 days, every 0.5-1.5, every 1-2, or every 1-1.5 days of the culturing. In some embodiments, the increasing is effected every 20-28 hours of the culturing. In some embodiments, the increasing is effected at daily intervals. In some embodiments, the oxygen concentration is incrementally increased from about 5% to about 12% during the culturing process at daily intervals. For example, the initial oxygen concentration is about 5% at the beginning of the culturing process (e.g., at day 5), which can be increased incrementally at, for example, 1% per day until the oxygen level reaches 12% at day 12. In some embodiments, the culture condition used in culturing the pre-implantation embryo in the perfusable vascularized tissue or in the uterus-like tissue structure does not comprise decreasing the oxygen concentration throughout the culturing.

Similarly, the culture chamber can have an atmosphere comprising an increasing gas pressure from about 2 to about 5 pounds per square inch (psi). The gas pressure can be increased throughout the culturing starting from about 2 psi, to 2.5 psi, 3 psi, 3.5 psi, 4 psi, 4.5 psi, 5 psi or higher at any suitable time interval (e.g., daily interval). In some embodiments, the increasing of gas pressure is effected every 0.5-2 days, every 0.5-1.5, every 1-2, or every 1-1.5 days of the culturing. In some embodiments, the increasing is effected every 20-28 hours of the culturing. In some embodiments, the increasing is effected at daily intervals. For example, the initial gas pressure can be about 2 psi at the beginning of the culturing process (e.g., at day 5), which can be incrementally increased at, for example, 0.5 psi per day until the gas pressure reaches 5 psi at day 12. In some embodiments, the culture condition used in culturing the pre-implantation embryo in the perfusable vascularized tissue or in the uterus-like tissue structure does not comprise decreasing the gas pressure throughout the culturing.

In some embodiments, culturing a human embryo at pre-implantation stage in the perfusable vasculature tissue or the uterus-like tissue structure comprises providing an in vitro culture media to the perfusable vasculature tissue or to the uterus-like tissue structure wherein the in vitro culture media is suitable for supporting the growth, in vitro implantation, and further development of the human embryo. In some embodiments, the embryo is cultured in a microfluidic device described herein above, and the in vitro culture media can be supplied to the embryo in the exposed chamber, e.g., via the opening (punch out) in the exposed chamber of the microfluidic device. In some embodiments, the in vitro embryo culture media is not perfused, but added to the exposed chamber from the wells in the top plate which are fluidly connected to the punch out.

The in vitro culture media used herein for culturing the human embryo in the perfusable vascularized tissue or in the uterus-like tissue structure comprises a basal culture medium. The basal medium may comprise water, salts, amino acids, a carbon source, vitamins, lipids and a buffer. Suitable carbon sources may be assessed by one of skill in the art from compounds such as glucose, sucrose, sorbitol, galactose, mannose, fructose, mannitol, maltodextrin, trehalose dihydrate, and cyclodextrin. The basal culture medium can comprise Dulbecco's Modified Eagle Medium (DMEM), DMEM Nutrient Mixture 12 (DMEM/F12), a non-human serum or serum substitute thereof, an antibiotic, L-glutamine or an analogue thereof (e.g., GlutaMAX™), or any combination thereof.

The non-human serum or serum substitute can comprise fetal bovine serum, bovine serum albumin, rat serum, KnockOut™ Serum Replacement, or any combination thereof. The antibiotic can comprise Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. In some embodiments, the culture media comprises a reducing agent. The reducing agent can comprise beta-mercaptoethanol (BME), N-acetyl-L-cysteine, dithiothreitol (DTT), or any combination thereof.

The concentration or amount of one or more of the components in a solution or media can vary. The amount of, e.g., the non-human serum or serum substitute thereof, antibiotic, reducing agent, and/or L-glutamine (e.g., GlutaMax™) can vary, and, in some embodiments, can be adjusted as needed by one of skill in the art. In some embodiments, the amount of non-human serum or serum substitute thereof can comprise about 0.01% to about 40% (e.g., about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the amount of antibiotic can comprise about 0.01% to about 10% (e.g., about 0.01%, 0.1%, 1%, 2%, 3%, 4%, 5%, 6%, 7%, 8%, 9%, 10% or a number or a range between any two of these values) volume per volume (% v/v), weight per volume (% w/v) or weight per weight (% w/w) of the medium. The amount of e.g., the reducing agent can vary. For example, in some embodiments, the concentration of the reducing agent in the composition can be about 0.1 μM to about 1 mM (e.g., about 0.1, 0.2, 0.3, 0.4, 0.5, 0.6, 0.7, 0.8, 0.9, 1, 10, 100, 200, 300, 400, 500, 600, 700, 800, 900 μM, 1 mM, or a number or a range between any two of these values). The amount of L-glutamine (e.g., GlutaMAX™) can vary. For example, in some embodiments, the concentration of L-glutamine in the culture media can be about 0.1 mM to about 40 mM, about 0.2 mM to about 20 mM, about 0.5 mM to about 10 mM, about 1 mM to about 5 mM or about 1.5 mM to about 2.5 mM e.g., about 2 mM. Where percentages are provided for agents, ingredients and compounds, they can be % w/w, % w/v or % v/v with respect to the formulation as a whole, unless otherwise indicated.

In some embodiments, the in vitro culture media comprises non-essential amino acids. Non-essential amino acids may be included in the culture medium, for example, comprising glycine (about 1 mg/ml to about 25 mg/ml or about 5 mg/ml to about 10 mg/ml e.g., about 7.5 mg/ml), L-alanine (about 1 mg/ml to about 25 mg/ml or about 5 mg/ml to about 10 mg/ml e.g., about 9 mg/ml), L-asparagine (about 5 mg/ml to about 30 mg/ml or about 10 mg/ml to about 15 mg/ml e.g., about 13.2 mg/ml), L-aspartic acid (about 5 mg/ml to about 30 mg/ml or about 10 mg/ml to about 15 mg/ml e.g., about 13 mg/ml), L-glutamic acid (about 5 mg/ml to about 50 mg/ml or about 10 mg/ml to about 20 mg/ml e.g., about 15 mg/ml), L-proline (about 5 mg/ml to about 30 mg/ml or about 10 mg/ml to about 15 mg/ml e.g., about 11 mg/ml) and/or L-serine (about 5 mg/ml to about 30 mg/ml or about 10 mg/ml to about 15 mg/ml e.g., about 11 mg/ml).

In some embodiments, the in vitro culture media can further comprise an effective amount of sodium pyruvate, such as, for example, at a concentration of about 0.05 mM to about 20 mM (e.g., about 0.05, 0.1, 0.2, 0.3, 0.4, 0.5, 1, 1.5, 2, 2.5, 3, 3.5, 4, 4.5, 5, 6, 7, 8, 9, 10, 11, 12, 13, 14, 15, 16, 17, 18, 19, 20 mM or a number or a range between any two of these values).

Each component of the culture medium described herein may be present in an amount such that the culture medium is suitable for supporting the growth of a human embryo at pre-implantation stage to develop into a post-implantation embryo structure and/or further development of the post-implantation embryo structure.

In some embodiments, the in vitro culture media can further comprise (a) insulin, an insulin analogue, or an insulin receptor agonist; (b) estrogen, an estrogen analogue, or an estrogen receptor agonist; and (c) progesterone, a progesterone analogue, or a progesterone receptor agonist. In some embodiments, the insulin receptor agonist is selected from the group comprising IGF-I, IGF-II, analogues thereof, or any combination thereof. The estrogen receptor agonist can be selected from the group comprising β-estradiol, estrone, estriol and estetrol, or any analogue thereof. In some embodiments, the in vitro culture media comprises N-acetylcysteine (NAC) due to its antioxidant properties and ability to influence cell behavior.

In some embodiments, culturing the human embryo at pre-implantation stage in the perfusable vascularized tissue or in the uterus-like tissue structure comprises increasing serum concentrations in the culture media, optionally increasing the serum concentrations from about 10% to about 30%. Accordingly, the serum concentrations is provided in the in vitro culture media at increasing concentrations throughout a dynamic condition. In some embodiments, the in vitro culture media comprises fetal bovine serum, and the concentration of the fetal bovine serum can be increased from about 10% to about 30%.

The serum concentrations can be increased throughout the culturing starting from about 10%, to 12%, 14%, 16%, 18%, 20%, 22%, 24%, 26%, 28%, 30%, or higher at any suitable time interval. In some embodiments, the increasing of serum concentration is effected every 0.5-2 days, every 1-1.5, every 1-2, every 2-3 days of the culturing. In some embodiments, the increasing is effected every 48-72 hours of the culturing. In some embodiments, the serum concentration (e.g., fetal bovine serum) is increased from about 10% to about 30% during the culturing process. For example, the initial serum concentration is about 10% at the beginning of the culturing process (e.g., at day 5 and/or day 6), which can be increased progressively at, for example, 10% every two or three days until the serum concentration reaches 30% at day 12. In an exemplary embodiment, the fetal bovine serum is maintained at about 10% for two days (day 6-7), at about 20% for three days (day 8-10), and at about 30% for two days (day 11-12). In some embodiments, the culture condition used in culturing the pre-implantation embryo in the perfusable vascularized tissue or in the uterus-like tissue structure does not comprise decreasing the serum concentration throughout the culturing.

Prior to culturing in the perfusable vascularized tissue or in the uterus-like tissue structure, the embryo can be cultured at any in vitro culture media under a condition suitable for the growth and development of the embryo as would be understood by a person skilled in the art. In some embodiments, the human embryo is cultured in a global culture media prior to culturing in the perfusable vascularized tissue or in the uterus-like tissue structure, optionally for 1, 2, 3, 4, or 5 days, and optionally wherein the oxygen concentration is maintained at about 5% and the gas pressure at about 2 psi. In some embodiments, the global culture media comprises at least 5% serum concentration. The serum can be a human serum albumin.

In an exemplary embodiment, culturing a human embryo at pre-implantation stage in the perfusable vascularized tissue in a culture media under a condition comprises transferring a human embryo at day 5 after fertilization (e.g., Carnegie stage 3-2) to the perfusable vascularized tissue at day 6, embedding the human embryo to the perfusable vascularized tissue at day 8, and culturing the human embryo in the perfusable vascularized tissue until at least day 12 after fertilization. The serum concentrations in the culture media, oxygen concentrations, and gas pressures in the culture chamber are incrementally increased during the culturing. The serum concentrations are maintained at about 10% for two days (day 6-7), at about 20% for three days (day 8-10), and at about 30% for two days (day 11-12). The initial oxygen concentration is about 5% at the beginning of the culturing process (at day 5), which is increased incrementally at 1% per day until the oxygen level reaches 12% at day 12. The initial gas pressure is about 2 psi at the beginning of the culturing process (at day 5), which is incrementally increased at about 0.5 psi per day until the gas pressure reaches 5 psi at day 12. The culture condition used herein does not comprise decreasing the oxygen concentration, the gas pressure, nor the serum concentration throughout the culturing.

The post-implantation embryos generated herein exhibit three-dimensional post-implantation morphological features similar to natural embryo structures. Morphological features include, but not limited to, the formation of amniotic cavity, primary yolk sac cavity, chorionic cavity, lacunae in the syncytiotrophoblast region, and/or migrating trophoblast cells. Analysis of a Day 12 embryo indicates the presence of cell types such as amnion, primordial germ cells, visceral endoderm and parietal endoderm cells. An extensive trophoblast vascular interaction and vessel dilation are also observed in the Day 12 embryos. In some embodiments, the synthetic embryo generated herein resembles a Carnegie stage 5 (c).

In some embodiments, the embryo at the post-implantation stage can comprise NANOG+ epiblast cells cavitating to give rise to an amniotic cavity, SOX17+ hypoblast cells forming a primary yolk sac, and AP2-Gamma (AP2Y)+ trophoblast cells forming a trophoblast region. In some embodiments, the mammalian embryo at the post-implantation stage comprises primary yolk sac with SOX17+ visceral and parietal endoderm cells, a chorionic cavity adjacent to the yolk sac, NANOG+SOX17+AP2-gamma+ primordial germ cells, and/or migratory trophoblast cells that interact with endothelial vessels to cause vascular mimicry, vasodilation and angiogenesis. Specifically, some trophoblast cell are positioned behind an endothelial cell forming the blood vessel, while other trophoblast cell can replace endothelial cells to become part of the vascular lining and exhibit CD31 expression indicating vascular mimicry. Absolute values of HCG and (Interleukin-6) IL-6 in the media circulating within the vasculature starting from Day7 of culture in the vascularized platform described herein (or 9 days post fertilization) are measured. This indicates functional interaction between trophoblast and vasculature. HCG is a hormone released by trophoblast cells of the embryo post-implantation and IL-6 is a cytokine released by trophoblast cells that serves a myriad of functions, such asm, but not limited to immune modulation and vascular remodeling.

In some embodiments, the embryos used herein for culturing in the synthetic tissues (e.g., perfusable vascularized tissue or uterus-like tissue) are blastoids or blastocyst-like cell aggregates. As used herein, the terms “blastoids”, “blastocyst-like cell aggregates”, or “blastocyst-like structure” are used interchangeably to reflect the tissues that model blastocyst without being true blastocyst. Blastoids can recapitulate the three-dimensional morphological and molecular signatures of the human blastocyst including the concomitant specification and spatial organization of tissues reflecting the three founding lineages that form the whole organism, namely the trophectoderm, the epiblast and the hypoblast. Blastoids and blastocyst-like cell aggregates can be generated by culturing an aggregate of human pluripotent stem cells and trophoblast cells in a suitable culture medium. Methods for generating blastoids or blastocyst-like cell aggregates are known in the art. For example, the blastoids or blastocyst-like cell aggregates are generated using the methods and culture media described in CA3204537A1, the content of which is incorporated herein by reference in its entirety.

Post-implantation blastoids generated using the method described herein exhibit morphological features similar to natural human embryos at equivalent stages. In some embodiments, post-implantation blastoids exhibit functional trophoblast-vascular interaction indicated by the presence of human chorionic gonadotropin (hCG) in the vascular media. Morphological features include, but not limited to, the formation of amniotic cavity, primary yolk sac cavity, lacunae in the syncytiotrophoblast region, and/or migrating trophoblast cells.

In some embodiments, the embryos used herein for culturing in the synthetic tissues (e.g., perfusable vascularized tissue or uterus-like tissue) are synthetic embryos generated in vitro from pluripotent stem cells such as mammalian embryonic stem cells alone or together with extra-embryonic stem cells. The embryonic stem cells may be wild type embryonic stem cells or inducible embryonic stem cells capable of expressing a GATA transcription factor upon induction. The extra-embryonic stem cells can comprise a trophoblast stem cell, an inducible extra-embryonic endoderm stem cell, or both. Methods for generating mammalian embryos in vitro can be found example, US20220308041A1, US20240124835A1, US20240093144A1, and WO2023114754A1, the contents of which are incorporated herein by reference in their entireties.

In some embodiments, the embryos described herein are mammalian embryos. In some embodiments, the mammalian embryos are non-human embryos, such as mouse embryos or rabbit embryos. In some embodiments, the mammalian embryos are human embryos.

Embryonic stages of the synthetic embryos described herein can be assessed compared to an in vivo or natural embryo counterpart at the same developmental stage by multiple ways including, but not limited to, morphology, length, weight, weight, expression of developmental marker genes using specific antibodies or primers, transcriptional profiling and the like, as further described herein below and in the Examples section. Morphology assessment of embryonic development can be performed by previously established morphological features such as described in Carnegie stages of development (Also, See, Table 1; Developmental stages in human embryos. R. O'Rahilly and F. Muller (eds), Carnegie Institution of Washington, Washington, D C, 1987), in Theiler stages of development (see, for example, Table 2; www. emouseatlas org) or according to embryonic days.

In some embodiments, one or more developmental markers as described herein can be used to assess the developmental stage of a synthetic embryo structure. Numerous methods exist in the art for detecting the presence, absence, or amount of a marker gene product (e.g., mRNA and/or protein), as well as its localization in an embryo structure or subcellular localization (e.g., nucleus and/or cytoplasm). Marker expression may be assessed by any of a wide variety of well-known methods for detecting expression of a transcribed molecule or a protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification and sequencing methods.

In some embodiments, activity of a particular gene is characterized by a measure of gene transcript (e g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a marker and functionally similar homologs thereof, including a fragment or genetic alteration thereof (e.g., in regulatory or promoter regions thereof) comprises detecting or determining RNA levels for the marker of interest. In some embodiments, one or more cells from the synthetic embryo structure can be obtained and RNA is isolated from the cells. In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated. It is also be possible to obtain cells from, e.g., the synthetic embryo cells and culture the cells in vitro, such as to obtain a larger population of cells from which RNA can be extracted. Methods for establishing cultures of non-transformed cells (primary cell cultures) are known in the art. In some embodiments, cells can be dissociated (e.g., by enzymatic or mechanical means), and isolated by methods known in the art (e g., Fluorescence-Activated Cell Sorting, Microfluidics, etc.)

When isolating RNA from, e.g., synthetic embryos at various developmental stages and/or cells comprising said synthetic embryos, it may be important to prevent any further changes in gene expression after the tissue or cells has been removed from the subject. Changes in expression levels are known to change rapidly following perturbations, e g., heat shock or activation with lipopolysaccharide (LPS) or other reagents. In addition, the RNA in the tissue and cells may quickly become degraded. Accordingly, in some embodiments, the tissue or cells obtained from a subject is snap frozen as soon as possible.

RNA can be extracted from cells by a variety of methods, e.g., the guanidium thiocyanate lysis followed by CsCl centrifugation. Methods for obtaining RNA from single-cells are also known in the art. The RNA sample can then be enriched in particular species. In some embodiments, poly(A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.). In some embodiments, the RNA population is enriched in marker sequences. Enrichment can be undertaken, e.g., by primer-specific cDNA synthesis, or multiple rounds of linear amplification based on cDNA synthesis and template-directed in vitro transcription.

The population of RNA, enriched or not in particular species or sequences, can further be amplified. As defined herein, an “amplification process” increases the number of copies of a polynucleotide (e.g., RNA). For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the disclosed methods to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4:80-84 (1994). Real time PCR may also be used. Other known amplification methods which can be utilized herein include but are not limited to the so-called “NASBA” or “3 SR” technique described in PNAS USA 87:1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42:9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e g., Kwoh et al., Proc. Natl. Acad. Sci. USA 86, 1173 (1989)). Many techniques are known in the state of the art for determining absolute and relative levels of gene expression, commonly used techniques suitable for use in the disclosed methods include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Nonradioactive labels such as digoxigenin may also be used. In some embodiments, the probe is labeled with a fluorescence moiety.

Alternatively, mRNA expression can be detected on a DNA array, chip or a microarray. Labeled nucleic acids of a test sample obtained from a subject may be hybridized to a solid surface comprising marker DNA. Positive hybridization signal is obtained with the sample containing marker transcripts. Methods of preparing DNA arrays and their use are well known in the art (see, e.g., U.S. Pat. Nos. 66,186,796; 6,379,897; 6,664,377; 6,451,536; 548,257; U.S. 20030157485). Serial Analysis of Gene Expression (SAGE) can also be performed (See for example U.S. patent application No. 20030215858). In some embodiments, next generation sequencing (e.g., RNA-seq) can be used to analyze total mRNA expression from one (e.g., single-cell RNA-seq) or more cells. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLID sequencing, and nanopore sequencing amongst others. Methods for constructing sequencing libraries are known in the art.

In some embodiments, the single cell sequencing can be high-throughput single cell RNA sequencing. In some embodiments, the single cell sequencing is a low cost high-throughput single cell RNA sequencing. Not being bound by any particular theory, the single cell RNA sequencing is capable of efficiently and cost effectively sequencing thousands to tens of thousands of single cells. In some embodiments, single cell RNA sequencing comprises pairing single cells in droplets with oligonucleotides for reverse transcription, wherein the oligonucleotides are configured to provide cell-of-origin specific barcodes uniquely identifying transcripts from each cell and a unique molecular identifier (UMI) uniquely identifying each transcript. In some embodiments, single cell RNA sequencing comprises pairing single cells in droplets with single microparticle beads coated with oligonucleotides for reverse transcription, wherein the oligonucleotides contain a bead-specific barcode uniquely identifying each bead and a unique molecular identifier (UMI) uniquely identifying each primer. In some aspects of the disclosure, unbiased classifying of cells in a biological sample comprises sequencing the transcriptomes of thousands of cells, preferably tens of thousands of cells (e.g., greater than 1000 cells, or greater than 10,000 cells).

The activity or level of a lineage marker protein can be detected and/or quantified by detecting or quantifying the expressed polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binderligand assays, immunohistochemical techniques, agglutination, complement assays, high performance liquid chromatography (HPLC), thin layer chromatography (TLC), hyperdiffusion chromatography, and the like.

Described below are non-limiting examples of techniques that may be used to detect marker protein according to a practitioner's preference based upon the present disclosure. One such technique is Western blotting, wherein a suitably treated sample is run on an SDS-PAGE gel before being transferred to a solid support, such as a nitrocellulose filter. Anti-marker protein antibodies (unlabeled) are then brought into contact with the support and assayed by a secondary immunological reagent, such as labeled protein A or anti-immunoglobulin (suitable labels including 1251, horseradish peroxidase, alkaline phosphatase, fluorophore). Chromatographic detection may also be used.

Immunohistochemistry may be used to detect expression of marker protein. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-marker protein antibodies, such as intrabodies, may also be used for imaging purposes, for example, to detect the presence of marker protein in cells or, e.g., an EP structure. Suitable labels include radioisotopes, iodine (¹²⁵I, ¹²¹I), carbon (¹⁴C), sulphur (³⁵S), tritium (³H), indium (¹¹² In), and technetium (⁹⁹mTc), fluorescent labels, such as fluorescein and rhodamine, and biotin.

Antibodies that may be used to detect marker protein include any antibody, whether natural or synthetic, full length or a fragment thereof, monoclonal or polyclonal, that binds sufficiently strongly and specifically to the marker protein to be detected. An antibody may have a Ka of at most about 10⁻⁶M, 10⁻⁷M, 10⁻⁸M, 10⁻⁸ M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the marker protein relative to other proteins, such as related proteins.

Antibodies are commercially available or can be prepared by methods known in the art. Antibodies and derivatives thereof that may be used encompass polyclonal or monoclonal antibodies, chimeric, human, humanized, primatized (CDR-grafted), veneered or single-chain antibodies as well as functional fragments (marker protein binding fragments) of antibodies. For example, antibody fragments capable of binding to a marker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain. In some embodiments, agents that specifically bind to a marker protein other than antibodies are used, such as peptides. Peptides that specifically bind to a marker protein can be identified by any means known in the art. For example, specific peptide binders of a marker protein can be screened for using peptide phage display libraries.

The method can also comprise recording a plurality of images of the embryos. The plurality of images may be recorded over a pre-determined period of time, thus illustrating the development from, e.g., the pre-implantation stage to the post-implantation stage. The imaging apparatus may comprise microscopy apparatus, suitable recording apparatus, and optionally image processing apparatus.

Fluorescent markers, such as fluorescent dyes or fluorescent marker proteins, are used in the imaging of embryonic development. Such markers may be added to the culture system. For example, fluorescent dyes may be added to visualize particular molecules or cellular structures. For example, DAPI may be used to stain DNA or MitoTracker (Invitrogen) may be used to stain the mitochondria. Additionally or alternatively, synthetic embryo may produce such fluorescent markers endogenously, e.g., it may contain one or more cells which express a fluorescent marker protein. Such cells may have been genetically modified in order to confer the ability to express such a marker protein. Thus, fluorescence imaging apparatus may be particularly suitable for the methods described. The imaging apparatus may thus comprise a fluorescence microscope, such as a confocal microscope, that can include but is not limited to wide field, scanning and spinning disc confocal, and light sheet microscope.

Confocal microscopes image a single point of a specimen at any given time but allow generation of two dimensional or three dimensional images by scanning different points in a specimen in a regular raster to provide image data which can be assembled into a two or three dimensional image. For example, scanning a specimen in a single plane enables generation of a two dimensional image of a slice through the specimen. A plurality or “stack” of such two dimensional images can be combined to yield a three dimensional image. Spinning disc confocal microscopy provides added advantages over confocal laser scanning microscopy. Additionally, light sheet microscopy can also provide good imaging of embryonic development.

### Microfluidic Platform

Provided herein also includes a microfluidic platform. In some embodiments, the microfluidic platform can be used for preparing and culturing microtissues or cells such as the perfusable vascularized tissue, uterus-like structures, as well as mammalian embryos (e.g., human embryos) described herein.

The microfluidic platform provided herein can comprise a plurality of microfluidic device each having at least one tissue chamber for culturing cells or microtissues, wherein the at least one tissue chamber comprises an enclosed chamber, an exposed chamber accessible from a top plate via an opening, and interdigitated pillars at the interface between the enclosed chamber and the exposed chamber, the enclosed chamber in fluidic communication with the enclosed chamber, and wherein the enclosed chamber is fluidly connected to a media inlet and a media outlet, and the enclosed chamber and the exposed chamber each is connected to at least one port. In some embodiments, the one or more ports are for loading cell-hydrogel solution while the media inlet/outlet are for loading media suitable for culturing the vascularized tissue structures.

Similar to the microfluidic devices described in US Patents U.S. Pat. No. 9,810,685 B2 and U.S. Pat. No. 11,898,129 B2, the contents of which are incorporated herein by reference in their entireties, the microfluidic device described herein comprises a tissue chamber connected to two adjacent microfluidic channels through capillary burst valves (a small opening, also referred to as “perfusion microvalve”) that confine the loaded cell-hydrogel mixture inside the tissue chamber. Each tissue chamber, specifically the enclosed chamber, is connected to each adjacent microfluidic channel through one or more perfusion microvalve (e.g., 1, 2, or more). The number of the perfusion microvalves can be selected based on the width of the perfusion microvalve as well as the volume and flow rate of the medium supplied to the chamber. In some embodiments, the perfusion microvalve can have a width (or opening) of about 50 μm to about 100 μm (e.g., 50 μm, 60 μm, 70 μm, 80 μm, 90 μm, 100 μm, or a number or a range between any two of these values).

The design of fabricating adjacent microfluidic channels separated by a perfusion microvalve allows for adding a cell-hydrogel mixture into one microfluidic channel without the fluid bursting into the other adjacent microfluidic channels, so that when the hydrogel solidifies, it generates a well-controlled interface in the perfusion microvalves with adjacent microfluidic channels. This allows making perfusable vasculature in the first channel, which can be accessed from the adjacent second channel fluidly. Additional description about gel interface control at the perfusion microvalve can be found, for example, in U.S. Pat. No. 11,898,129, the content of which is incorporated herein by reference in its entirety.

In some embodiments, the two or more perfusion microvalves each has a burst pressure and is configured to have a first function of preventing hydrogel or hydrogel precursor from bursting into at least one of the microfluidic channels and a second function for perfusing nutrients through the tissue chamber including the enclosed chamber and the exposed chamber. In some embodiments, the perfusion microvalves are located in the enclosed chamber of the tissue chamber. The enclosed chamber is directly connected to adjacent microfluidic channels located parallel to the enclosed chamber via the perfusion microvalves. The microfluidic platform comprises a media inlet fluidly connected to the enclosed chamber on one side via one perfusion microvalve and a media outlet fluidly connected to the enclosed chamber on the other side via another perfusion microvalve (see, for example, FIG. 1C).

In some embodiments, the exposed chamber is configured for loading embryos and embryo culture media from the opening. The media inlet and/or outlet are configured for loading a vasculature media when preparing the perfusable vascularized tissue or for loading a minimum differentiation media when preparing a uterus-like tissue structure. The at least one hydrogel loading port is configured for loading hydrogel, hydrogel precursor, or a cell-hydrogel solution when preparing the perfusable vascularized tissue. In some embodiments, the enclosed chamber and the exposed chamber are seeded with endothelial cells, fibroblast cells, and a hydrogel for preparing a perfusable vascularized tissue. The endothelial cell, fibroblast cells, and hydrogel can be seeded to the enclosed chamber first, followed by seeding to the exposed chamber. The cell-hydrogel mixture in the enclosed chamber and in the exposed chamber can have the same or different height.

In some embodiments, the one or more tissue chamber can be fluidly connected to a pressure regulator module through a microfluidic channel. The pressure regulator module is provided that allows for reproducible gel loading without the risk of gel overflow into the microfluidic channels. In some embodiments, a pressure regulator module for a chip-based microfluidic platform can include a microfluidic channel including an inlet region and an outlet region downstream of the inlet region, said channel for passing flowable material from the inlet region through the outlet region and into a downstream compartment, one or more microvalves fluidly connected to the microfluidic channel and disposed upstream of the outlet region, said microvalves for releasing pressure in the microfluidic channel, wherein each microvalve has an open state permitting flowable material to pass through said microvalve and a closed state preventing flowable material from passing through said microvalve, and wherein each microvalve in its open state diverts flowable material from the microfluidic channel, and one or more reservoirs fluidly connected to the microvalves, for receiving flowable material diverted by the microvalves, wherein a flow of flowable material passing from the inlet region toward the downstream compartment is at least partially diverted by at least one of the microvalves into at least one of the reservoirs as a result of a pressure increase in the microfluidic channel. In some embodiments of the pressure regulator module, the one or more microvalves are capillary burst valves.

As used herein, a flowable material is a material that can flow in a microfluidic channel. Examples of flowable materials include, but are not limited to, liquids, cell culture media, cell suspensions, hydrogels, cell-hydrogel mixtures, blood, blood substitutes, and the like.

Any suitable hydrogel described herein can be loaded to the tissue chamber (the enclosed chamber and exposed chamber). Examples of hydrogels include, but are not limited to, collagen-based hydrogels, fibrin-based hydrogels, poly(ethylene glycol) (“PEG”)-based hydrogels, and the like.

Hydrogel can be prepared, for example, by warming of a collagen type-I solution to promote polymerization, by addition of biological accelerants such as thrombin to fibrinogen, or by photo cross-linking of polymers.

In some embodiments, cell-hydrogel mixtures can be prepared, for example, by warming a collagen type-I solution containing cells to promote polymerization, or by the addition of biological accelerants such as thrombin to mixtures of cells and fibrinogen.

Cells for use in cell-hydrogel mixtures, or for culturing in tissue compartments, include, but are not limited to, stem, endothelial, stromal, epithelial, immune, neuronal, connective, myocardial, hepato, renal, heart, liver, pancreas, muscle, brain, and kidney cells, and any kind of tumor cell. In some embodiments, combinations of these cells can be cultured or included in cell-hydrogel mixtures.

In some embodiments, as shown in FIG. 1A of U.S. Pat. No. 11,898,129 B2, the on-chip pressure regulator module consists of pressure-releasing capillary burst valves (denoted as safety microvalves) and diversion channels, all based on three basic design principles. First, the burst pressure of safety microvalves in the pressure regulator module should be lower than that of the tissue chamber capillary burst valves (denoted as perfusion microvalves) used for confining loaded gel inside the tissue chambers. This ensures that the safety microvalves will burst first to release redundant gel and regulate the pressure inside the tissue chambers to below the burst pressure of perfusion microvalves. Second, in order to redirect the redundant gel away from the tissue chambers, the pressure regulator module should be positioned upstream of the gel loading channel. Third, the volume of storage space to accommodate the redundant gel should be large enough for a typical single injection volume.

In some embodiments, a chip-based microfluidic platform described herein can comprise one or more compartments, with each compartment including one or more tissue chambers for culturing cells or microtissues, and the pressure regulator module fluidly connected by the outlet region to one or more of the compartments.

In some embodiments, a microfluidic platform can comprise a microfluidic chip containing a plurality of microfluidic devices (see e.g., FIG. 1A). Each microfluidic device can comprise a tissue chamber (see e.g., FIGS. 1B-C), wherein endothelial cells and fibroblast cells can be loaded with a suitable hydrogel described herein. Each tissue chamber can comprise two main chambers: an enclosed chamber and an exposed chamber. The enclosed chamber is a microfluidic channel that is enclosed within a base plate and not accessible from the top plate, while the exposed chamber is open to the top of the plate via a circular opening termed as a “punch out” providing access to the chamber from the top. The enclosed chamber are connected to two adjacent microfluidic channels through capillary burst valves (perfusion microvalves) that ensure when a cell-hydrogel mixture is added into the enclosed chamber (e.g., via port LI shown in FIG. 1C), the mixture does not burst into the media channels. Both the enclosed and exposed chambers are part of microfluidic channels and in fluidic communication with one another.

The tissue chamber, particularly the enclosed chamber, are connected to a media inlet and a media outlet (M1 and M2) that run along the device (FIGS. 1B-C). Additionally, the enclosed chamber and the exposed chamber each can be connected to at least one port. For example, the enclosed chamber can be connected to L1 shown in FIG. 1C and the exposed chamber can be connected to O1 and O2. In some embodiments, the port(s) connected to the enclosed chamber and the exposed chamber are different. This setup allows for the growth of a continuous and closed-loop vascular network in the hydrogel encompassing the two chambers. In some embodiments, ports such as L1, O1 and O2 are designed for loading the cell-hydrogel solution, while the media inlet/outlet are for supplying media such as the vasculature media.

The tissue chamber further comprises an interdigitated line of pillars (FIGS. 1B and 1D) at the interface between the enclosed chamber and the exposed chamber. Interdigitated pillars refer to a design where two sets of structure, resembling fingers, are arranged in an interlocked or interwoven manner, with spaces between them. Accordingly, when the cell-hydrogel mixture (endothelial cells, fibroblast cells and hydrogels) are seeded in the enclosed chamber, they can flow into the exposed chamber and partially fill it. The presence of the micropillars inside the tissue chamber also provides resistance to the flow of the cell-hydrogel when administered, e.g., via port L1. The cell-hydrogel is then added to the exposed chamber via ports O1 and O2, which, together with the punch out in the exposed chamber, allows the possibility to adjust the height of the cell-hydrogel in the exposed chamber.

The new design allows for a two-step process of building a perfusable vascularized tissue. In the first step, a cell-hydrogel mixture is administered into the enclosed chamber via port L1 which stops at the micropillars and does not burst into the media channels due to the presence of the perfusion microvalves. In the second step, the cell-hydrogel mixture is administered to the exposed chamber via ports O1/O2, generating a continuous cell-hydrogel solution spanning the enclosed chamber and the exposed chamber. The new design allows one to make perfusable vascularized tissue that is not only accessible via the fluidic channels in the enclosed area, but also from the exposed area via the punch out. For example, the vasculature media suitable for endothelial cell growth can be supplied through the fluidic channels (media inlet/outlet), while embryos or embryo culture media can be added directly to the chamber through the punch out to investigate their interaction and integration with the underlying vasculature.

In some embodiments, the microfluidic chip is placed under a top plate (FIG. 1A), and the ports and chambers can be accessed through the wells in the top place. In some embodiments, each implantation device unit occupies 6 wells in the top plate, and there are a total of 16 units lined up in the microfluidic chip, which occupies all 96 wells of the plate.

The chip-based microfluidic platform can be made using standard soft lithography methods or other microfabrication processes. A microfluidic device can be made, for example, of polydimethylsiloxane (PDMS) (Sylgard-184, Dow Corning) by micro-molding from SU-8 patterned silicon wafers using standard soft lithography techniques. A standard SU-8 photolithography process can be used to fabricate micro-molds. First, a layer of SU-8 can be spin-coated onto a Si-wafer (RCA-1 cleaned and 2% HF treated). Then, a single mask photolithography step can pattern a tissue chamber or microfluidic channel, for example. A PDMS layer can be molded on the micro-mold, then de-molded for further processing.

### EXAMPLES

Some aspects of the embodiments discussed above are disclosed in further detail in the following examples, which are not in any way intended to limit the scope of the present disclosure.

### Example 1

### Building 3-Dimensional Perfusable Vascularized Tissue Using a Microfluidic Device

This example presents the development of an in vitro platform to study the implantation and growth of human embryos and stem cell-base embryo models (FIGS. 1A-1D).

In an exemplary embodiment, the in vitro platform is a 96-well plate-like setup that encloses 16 microfluidic implantation devices (FIG. 1A). Each device comprises a tissue chamber (shown in blue in FIGS. 1B and 1C) where endometrial cells can be loaded with a suitable hydrogel. The tissue chamber comprises two main chambers: an enclosed chamber and an exposed chamber. The enclosed chamber is a microfluidic channel that is enclosed or embedded within the base plate and is connected to a media inlet and an outlet channel that run along the device (FIGS. 1B and 1C). The exposed chamber is open to the top of the plate via a circular opening (termed as punch out). Both the enclosed and exposed chambers are part of a microfluidic channel, separated by an interdigitated line of pillars (FIGS. 1B and 1D). Therefore, when the endometrial cells and hydrogels are seeded in the enclosed chamber, they can flow into the exposed chamber and partially fill it. This setup allows for the growth of a continuous and closed-loop vascular network in the hydrogel encompassing the two chambers.

To form the vasculature, a two-step protocol was followed. Step 1: Human umbilical cord vascular endothelial cells (HUVEC) and Human lung fibroblast (LF) cells were mixed with a hydrogel and seeded at a density of 3M cells/ml into the enclosed chamber via a port L1 (FIG. 1C). The total volume of the solution is about 4.5-5 μL. The cell-hydrogel solution stops at the pillars at the interface between the enclosed and exposed chamber in the device (FIG. 1B). Step 2:5 μL of the cell-hydrogel solution was seeded into the exposed chamber either via port C1 or C2 (FIG. 1C). This creates a continuous cell-hydrogel solution in both the enclosed and exposed chamber (FIG. 2). The seeding volume for step 2 can be increased to achieve a taller vascular bed in the exposed chamber.

The hydrogel used here is fibrin, which is fibrinogen cross-linked using thrombin at a ratio of 1:5. Fibrin hydrogel can be replaced by other types of natural hydrogels (e.g. Collagen 1, Hyaluronic acid, etc.) and synthetic hydrogel (e.g. PEG, PA, etc.). The fibrin hydrogel is functionalized with ECM proteins such as Laminin (20%) and Fibronectin (20%) to improve cell adhesion and growth of vasculature. Other ECM proteins such as Collagen-IV, Collagen-I and Hyaluronic acid (HA) can be supplemented to improve cell adhesion and survival.

Two cell types were used for this setup: endothelial cells and fibroblast cells. For the proof of concept, this example used commercially available primary endothelial cells derived from umbilical cord, Human umbilical cord vascular endothelial cells HUVEC cells and primary lung fibroblast (LF) cells. Other primary cell types such as endothelial progenitor cells derived from umbilical cord blood, mouse brain endothelial cells and immortalized cell lines such as EA.hy926 and HMEC-1 can also be used to generate the vascularized tissue. Similarly for fibroblast cells, human uterine fibroblast cells (HUF), human primary brain vascular fibroblast, mouse embryonic brain vascular fibroblast, human dermal fibroblast, human perivascular fibroblast can also be used with endothelial cells to generate the vascularized tissue.

After the fibrin hydrogel has solidified, 20 μL of Laminin solution is added into the media channels either via ports M1 or M2 (FIG. 1C) and incubated for 15 mins. The laminin solution is then replaced by supplemented EGM-2 media (Lonza CC-3162). EGM-2 media is purchased off the shelf that supports growth of vasculature. The primary components EGM-2 media and their respective functions of are: FBS (Fetal Bovine Serum)-2% final concentration, Hydrocortisone-modulates inflammation and promotes cell survival, Human Fibroblast Growth Factor-B (hFGF-B)-stimulates endothelial proliferation, Vascular Endothelial Growth Factor (VEGF)-promotes angiogenesis, R3-Insulin-like Growth Factor-1 (R3-IGF-1)-enhances growth, modified for stability, Ascorbic Acid (Vitamin C)-antioxidant and collagen synthesis support, Heparin-potentiates the activity of growth factors, Human Epidermal Growth Factor (hEGF)-stimulates proliferation, Gentamicin/Amphotericin B-antimicrobial.

A hydrostatic gradient of EGM-2 media is maintained in each device between ports M1 and M2 to generate media flow from the higher pressure to the lower pressure. Within 5-6 days, HUVEC and LF cells self-organize to form a closed-loop and continuous vasculature in the hydrogel encompassing the enclosed and exposed chambers. The LF cells acquire pericyte-like morphology and help stabilize vascular tubules (FIG. 2). The vasculature in the enclosed chamber anastomoses into the media inlet/outlet channels (FIG. 3A), which allows perfusion of media from the higher-pressure side to the lower-pressure side via the vasculature. The perfusion can be visualized by adding dye into the media. FIG. 3B shows the perfusion of a high-molecular density FITC Dextran dye (70 kDa) through the vessels indicating a perfusable and closed-loop vasculature.

### Example 2

### Implantation and Growth of Human Embryos

This examples investigates the implantation and growth of human embryos in the perfusable vascularized tissue generated in Example 1.

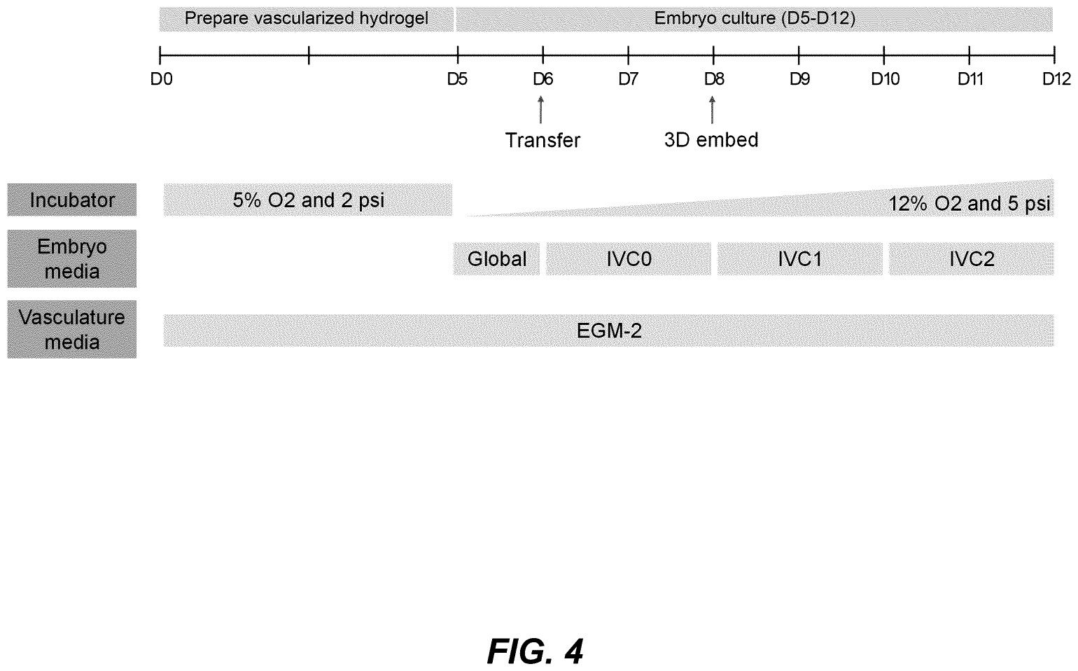

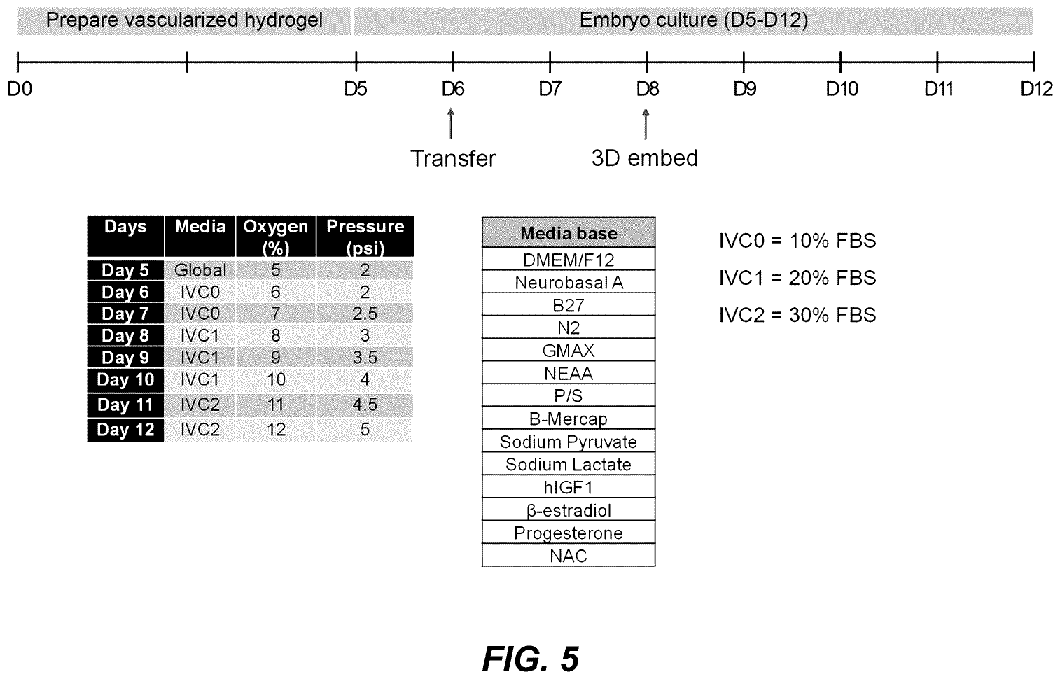

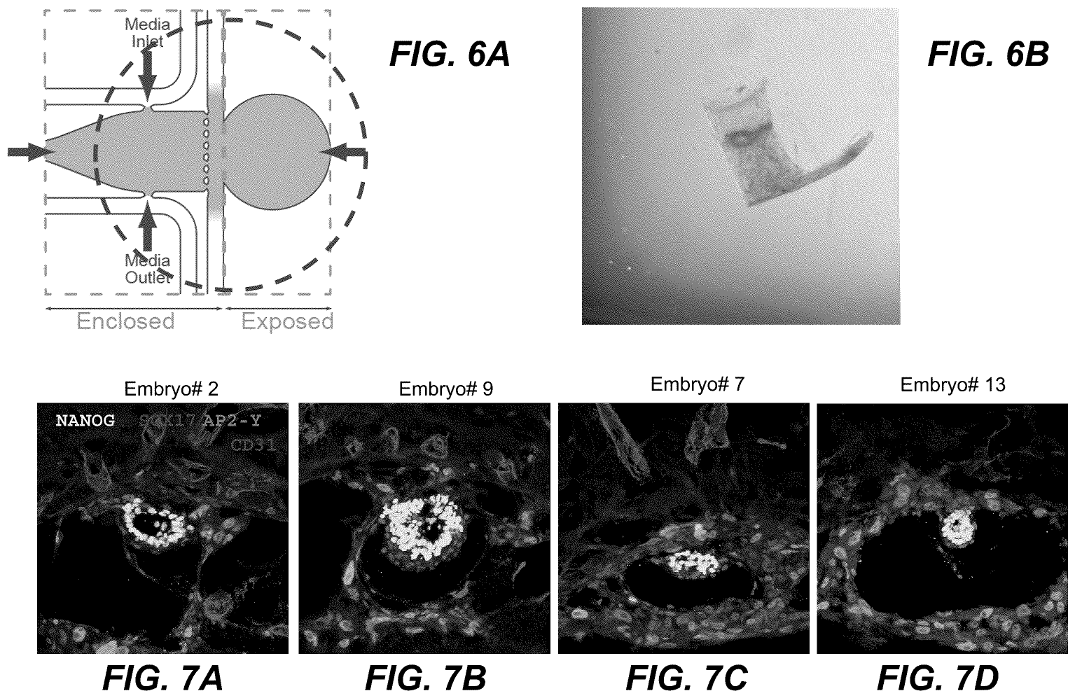

Human embryo was added to the exposed chamber of the device from Day5 (Carnegie Stage 3-2)-Day 12 (Carnegie Stage 5c) as show in FIG. 4 and FIG. 1B. A protocol was developed to dynamically modulate oxygen tension (percentage oxygen) and gas pressure (in psi) in the incubator that allows proper growth and stability of the vasculature and the embryo (FIG. 4). To precisely modulate oxygen and pressure, a commercially available incubator (Avatar from xcellbio) was used. A media and protocol were also developed to improve embryo growth (FIG. 5). The media comprises of a base media with progressive increase in fetal bovine serum (FBS) in it from 10% on D6 to 30% on D12. To avoid batch to batch variability, active hormones were stripped out of FBS using a dextran-charcoal-based heat-inactivation protocol (Rawlings, T. M., 2021, Modelling the impact of decidual senescence on embryo implantation in human endometrial assembloids. Elife, 10, e69603). The tissue can be dissected out of the device and processed for further analysis (FIG. 6B).

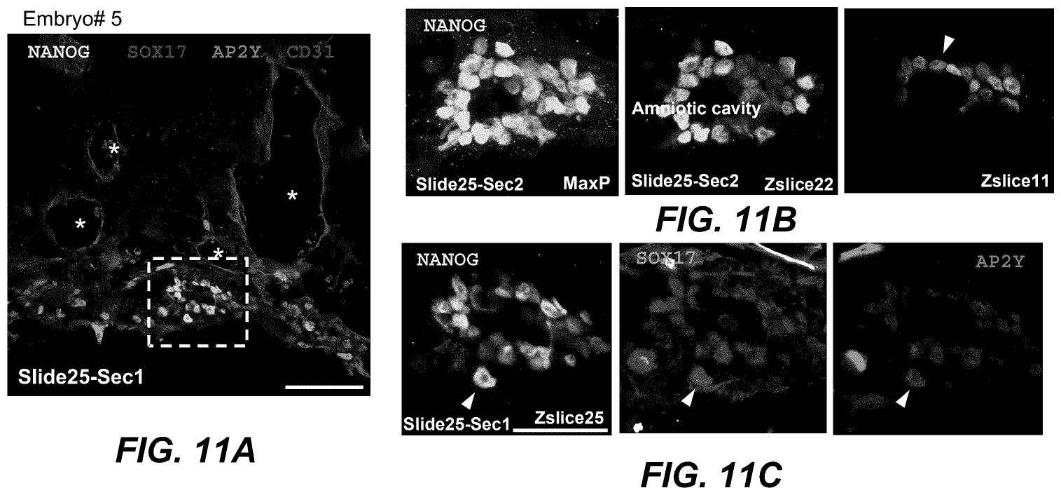

Human embryo at the blastocyst stage (Day5 or Carnegie Stage (CS) 3-2) was thawed and then transferred on top of the vascularized tissue in two-dimension (2D) or embedded in three-dimension (3D) alongside the vasculature. Best results were obtained when the blastocyst was transferred to the vasculature on D6 and embedded in the hydrogel on D8. Shown here are the results of 4 embryos that were cultured till Day10 (Carnegie stage 5b) and 1 embryo cultured till Day 12 (Carnegie stage 5c) (FIGS. 7A-7D and FIGS. 11A-11C). For Day10, the NANOGH epiblast cells (yellow) cavitate to give rise to the amniotic cavity, SOX17+ hypoblast cells (red) form the primary yolk sac (PYS) and AP2-Gamma (AP2Y)+ trophoblast (green) cells form the trophoblast region. The trophoblast cells were found in close proximity to the endothelial vessels (in red color marked by CD31/PECAM) (FIGS. 7A-7D). All 4 embryos were growing well and have acquired post-implantation morphology based on comparative analysis with corresponding Carnegie Stage (CS) 5b.

An exemplary study compared embryos cultured in the perfusable vascularized tissue described herein and that in natural uterine environment by assessing morphological features pertaining to individual lineages.

### Morphogenesis of the Epiblast Cells

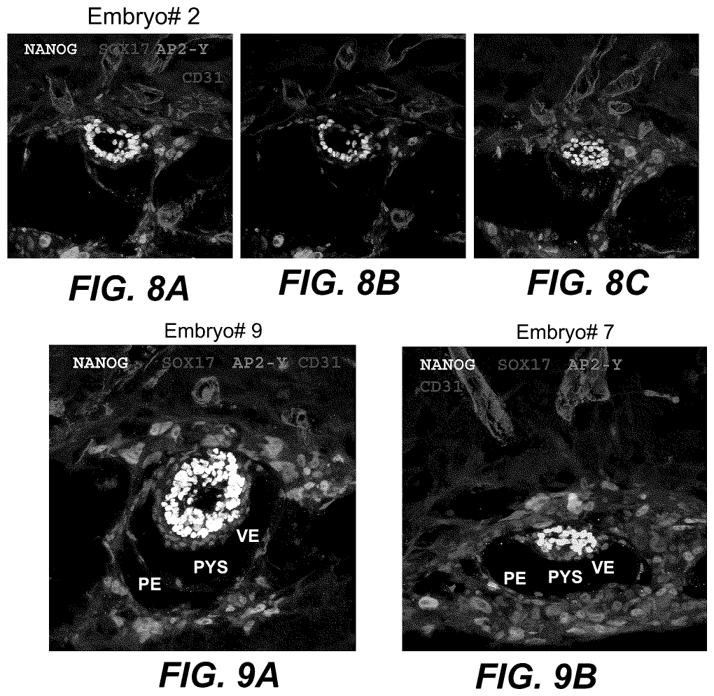

FIGS. 8A-8C show a montage of different sections of embryo #2 spanning the entire embryo and surrounding tissue. The human embryo in the perfusable vascularized tissue formed a bilaminar disc consisting of epiblast (NANOG+) that will give rise of the future embryo proper and the hypoblast that contributes to the yolk sac. The epiblast formed columnar epithelial layer and the hypoblast formed a cuboidal epithelial layer. A fluid-filled formed within the epiblast called the amniotic cavity (AC). The amniotic cavity separates the epiblast proper and the amnioblast, derived from the epiblast. The amnioblast (also NANOG+) has squamous morphology compared to the columnar morphology of the epiblast proper. The amnioblast is the progenitor cells for the future amniotic ectoderm.

### Morphogenesis of the Hypoblast Cells

FIGS. 9A-9B show the differentiation of the SOX17+ hypoblast cells and the formation of the primary yolk sac (PYS). Day10 (D10 or 10 days post fertilization (dpf)) embryo #9 and embryo #7 developed Primary Yolk Sac (PYS) enclosed by the visceral endoderm (VE) adjacent to the epiblast and parietal endoderm (PE). Visceral endoderm cells have higher density and exhibit cuboidal morphology compared to the parietal endoderm cells which are more squamous in morphology. NANOG+ cells indicate columnar epiblast, AP2-Y+ (TFAP2C) cells indicate trophoblast and CD31+ (PECAM) cells indicate endothelial blood vessels.

### Morphogenesis of the Primary Yolk Sac (PYS)

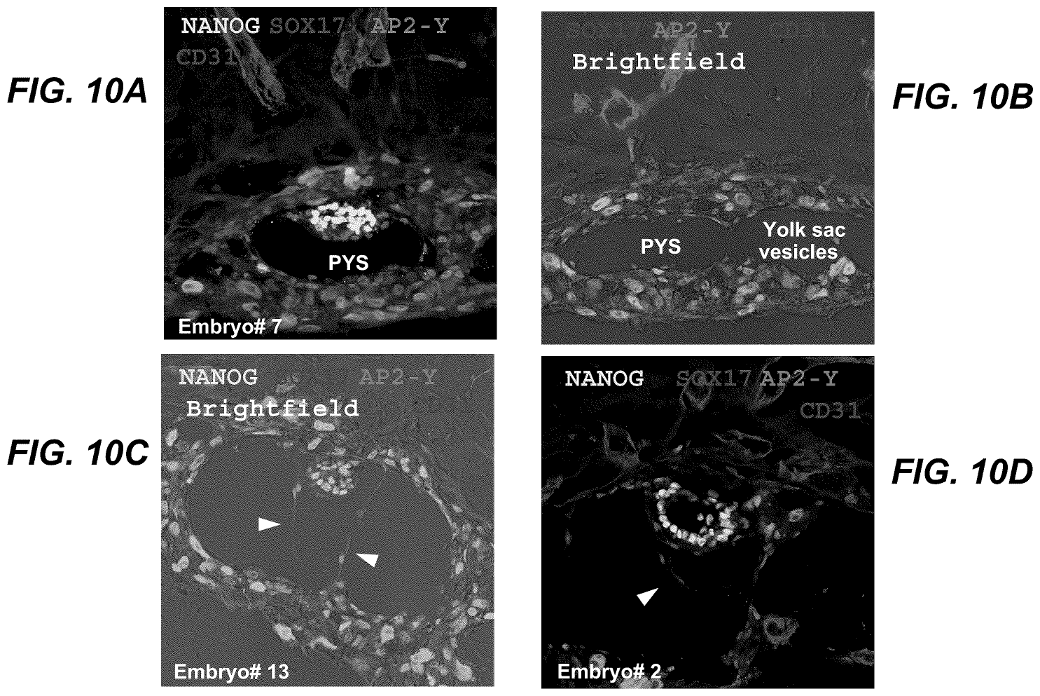

Analysis of D10 (or 10 dpf) embryo #7 reveals splitting of the PYS into smaller vesicles (FIGS. 10A-10D). Analysis of Embryo #2 and Embryo #13shows detachment of PYS from the trophoblast tissue as indicated by arrowheads. Analysis of natural human embryos at these stages shows similar features.

### Specification of the Amnion and Primodial Germ Cells (PGC)

FIGS. 11A-11C show that by D12 (12 dpf) NANOG+ epiblast cells gave rise to the extra-embryonic amnionic ectoderm (or Amnion) cells, which have a distinct squamous epithelial morphology compared to the columnar cuboidal morphology of epiblast cells. The epiblast cells also differentiated into primordia germ cells (PGCs) as indicated by the combined expression of NANOG+SOX17+AP2Y. Early PGC cells express these three reporters (Weatherbee, B. A., et al. (2023). Pluripotent stem cell-derived model of the post-implantation human embryo. Nature, 622 (7983), 584-593).

### Differentiation and Morphogenesis of Trophoblast Cells

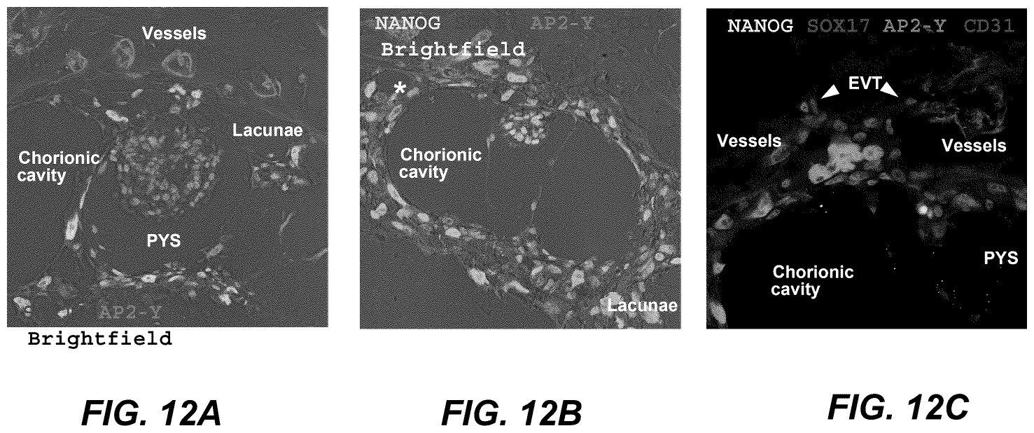

FIG. 12A shows that the trophoblast cells (AP2Y+) were in close proximity to the vasculature (CD31+) in the location of implantation. The trophoblast cells also formed fluid-filled cavities, which eventually merge to give rise to the chorionic cavity adjacent to the yolk sac. At the point of implantation, extra-villous trophoblast (EVT)-like cells can be visible migrating away from the embryo towards the blood vessels (C31+) (FIG. 12C).

### Trophoblast-Vascular Interaction

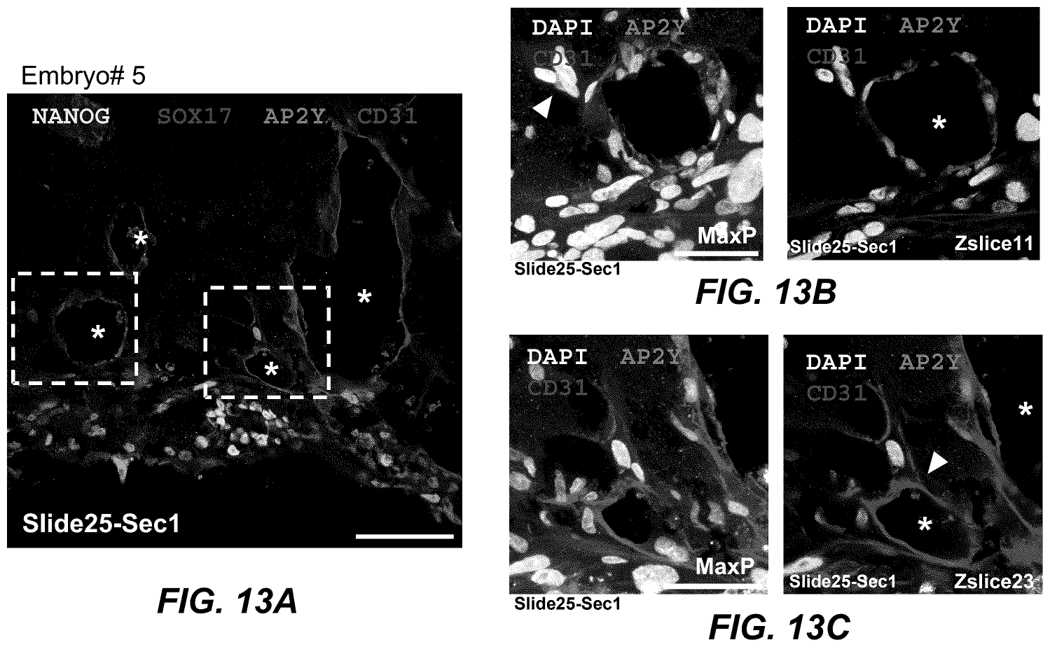

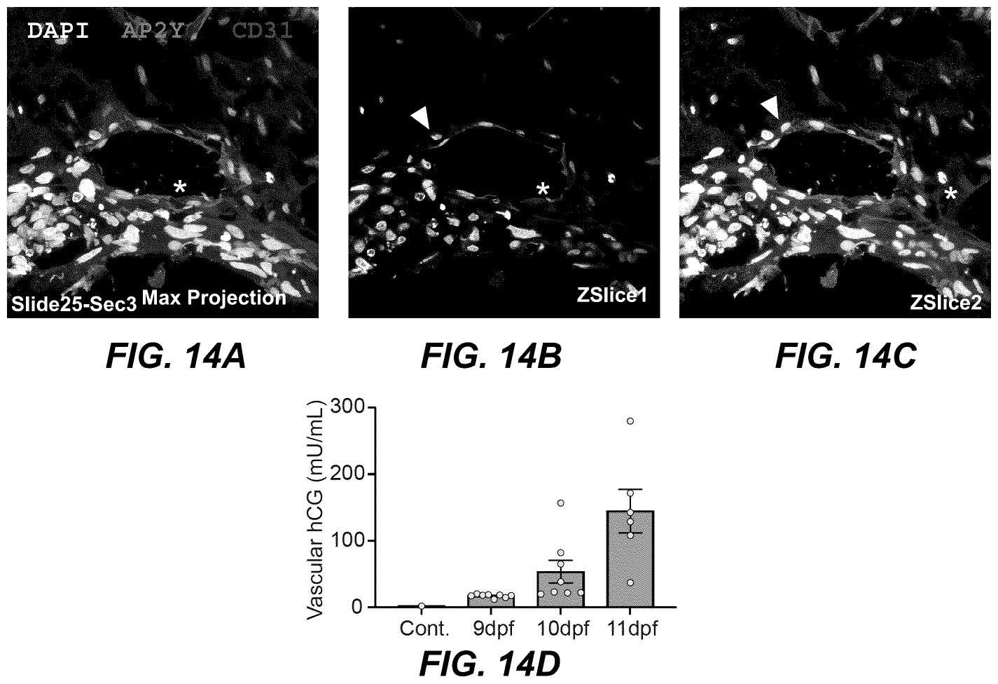

Trophoblast cells at the leading edge of the implantation can dilate maternal proximal blood vessels to enhance blood flow. In the present implantation model, by Day 12, blood vessels were significantly enlarged. Additionally, trophoblast cells (AP2Y+) cells can be found in proximity around blood vessels not far from the implantation area (FIGS. 13A-13C). FIGS. 14A-14C show trophoblast cells interacting with CD31+ endothelial vessel. Some trophoblast cell positioned behind an endothelial cell forming the blood vessel, while other trophoblast cell replaced endothelial cells to become part of the vascular lining and exhibit CD31 expression indicating vascular mimicry (FIG. 13C). These trophoblast cells could be endovascular EVTs that is well documented in mouse embryo development, but little is known about their behavior during these stages of human development. FIGS. 14A-14C shows that trophoblast invasion is deeperm blood vessels are larger, presence of extravillous trophoblast (EVT) and endovascular extravillous trophoblasts (EnEVT) cells, presence of Lacunae.

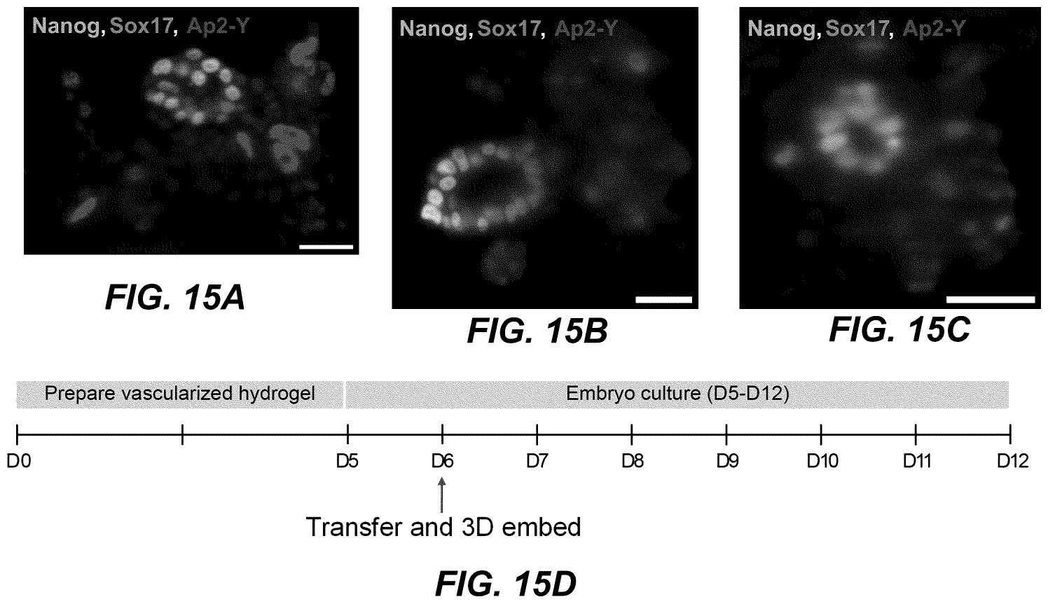

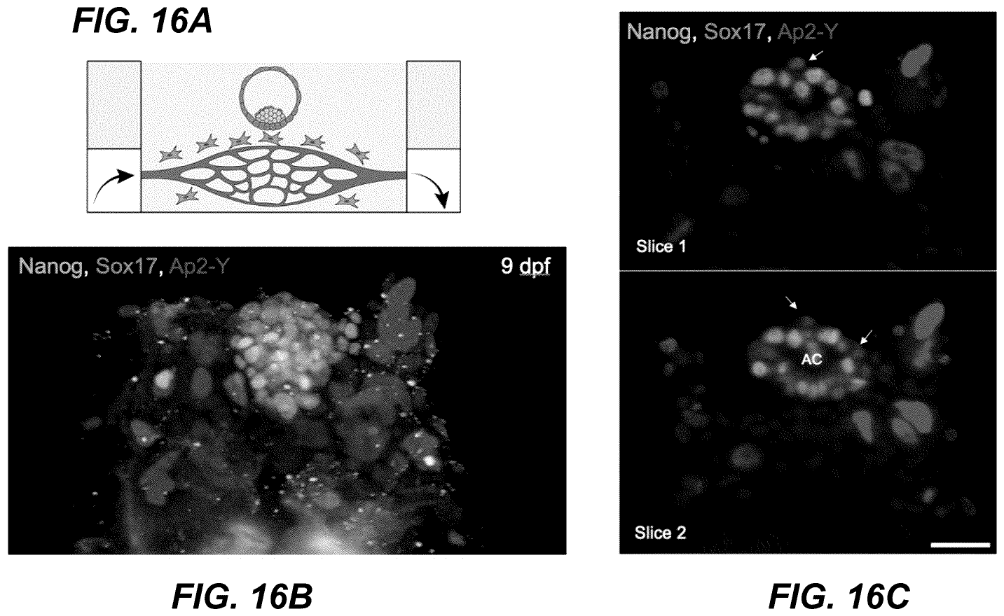

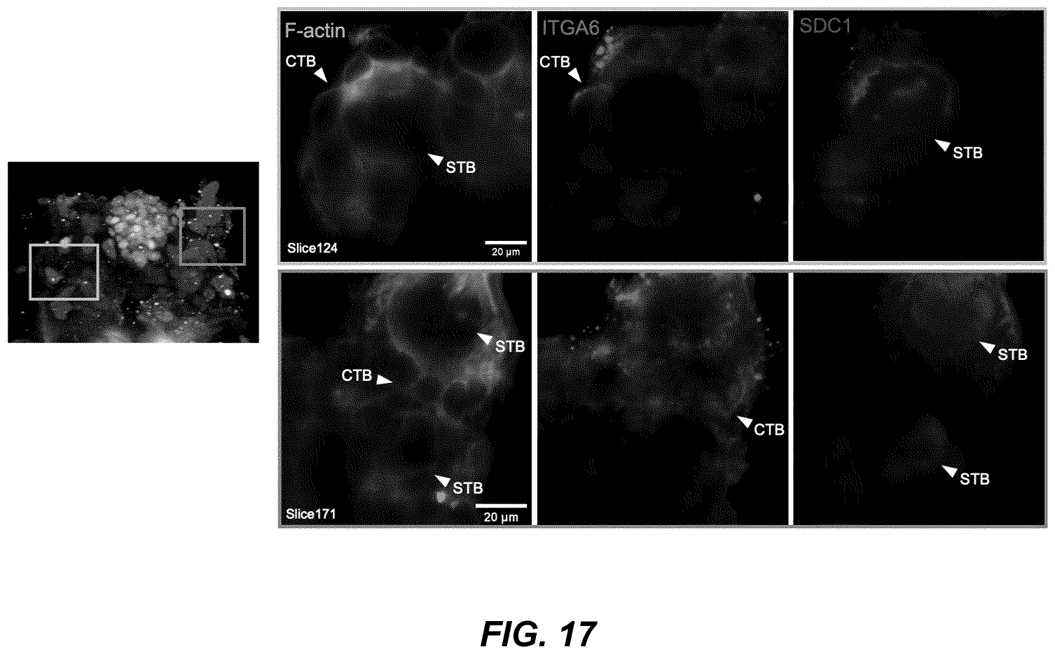

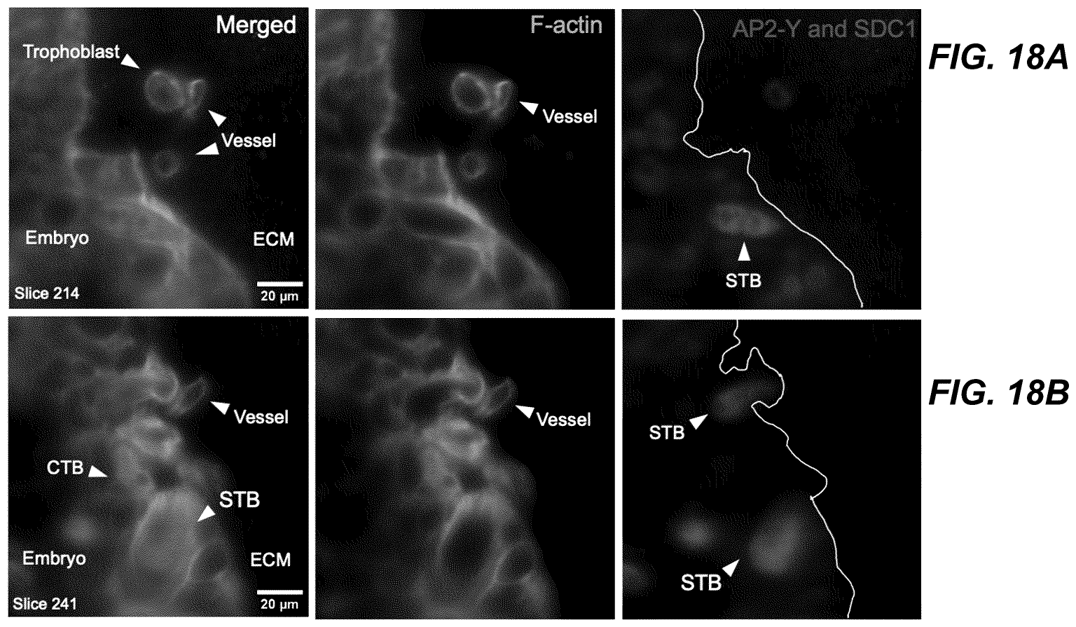

In another exemplary embodiment, embryos were cultured in the perfusable vascularized tissue generated in Example 1 but with a slightly different protocol (FIG. 15D) and morphological features pertaining to individual lineages were assessed. All the three embryos developed a NANOG+ epiblast with amniotic cavity. However, the SOX17+ hypoblast cells were diminished in number. In contrast to the experiments done in the above exemplary study, the embryos lack morphological features such as, the primary yolk sac, chorionic cavity and lacunae in the trophoblast region. Interestingly, the AP2-Y+ trophoblast cells (red) have proliferated extensively in 3D around the NANOG+ epiblast (green) and the SOX17+ hypoblast (blue) (FIGS. 16B, C and D). The trophoblast cells generated two distinct population of cells-cytotrophoblast (CTB, annotated as ITGA6) and syncytiotrophoblast (STB, annotated as SDC1) cells as shown in FIG. 17. The data suggests that the STB cells are being formed in between clusters of cytotrophoblast cells. Additionally, the AP2-Y+ trophoblast cells degrade the hydrogel and can be found in close physical proximity to the blood vessels (FIGS. 18A-B). These results highlight the importance of delayed hydrogel embedding on Day 8 compared to Day 6 to ensure proper growth and morphogenesis of the embryo.

### Example 3

### Implantation and Growth of Human Blastoid

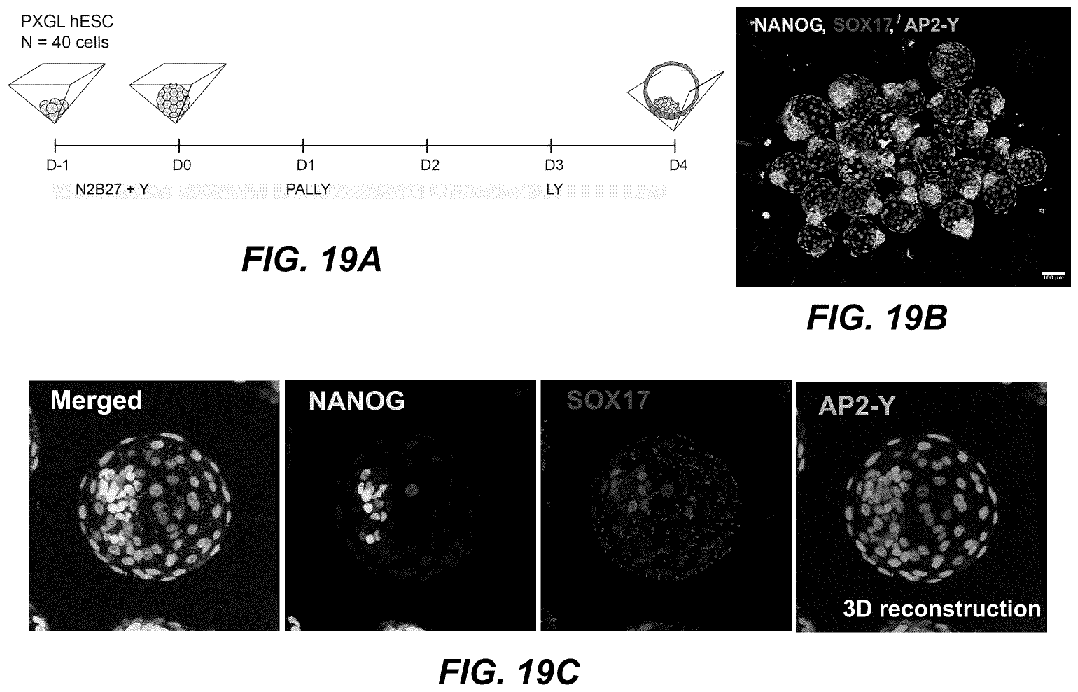

In this example, stem-cell derived blastocyst-like structures (called blastoids) were used to test for implantation and growth in the device. Blastoids were generated using a published protocol described in patent application number CA3204537A1, the content of which is incorporated herein by reference in its entirety (see FIG. 19A). This protocol can generate blastoids with the right proportion of the three lineages-NANOG+ epiblast, SOX17+ hypoblast and AP2-Y+ trophectoderm cells (FIGS. 19B-C). Day 4 blastoids have structural and morphological similarity with a Day 6-7 (6-7 dpf) natural embryo. Blastoids can be generated in large numbers which allows for testing various factors in optimizing the platform. However, blastoids have not been shown to develop properly like natural embryos in the post-implantation stages in vitro.

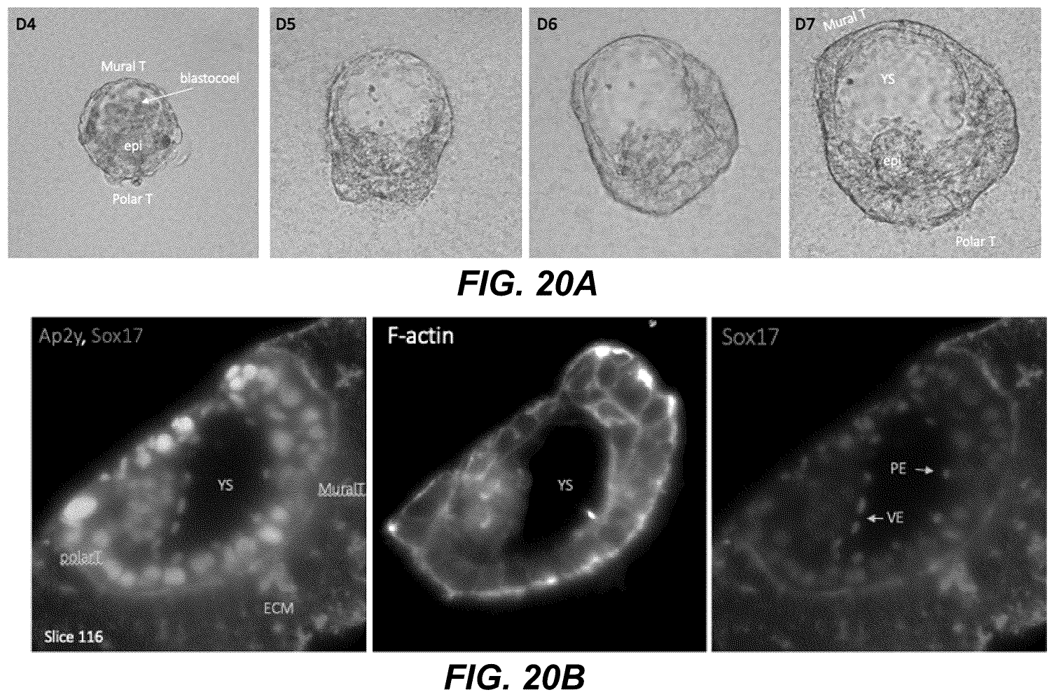

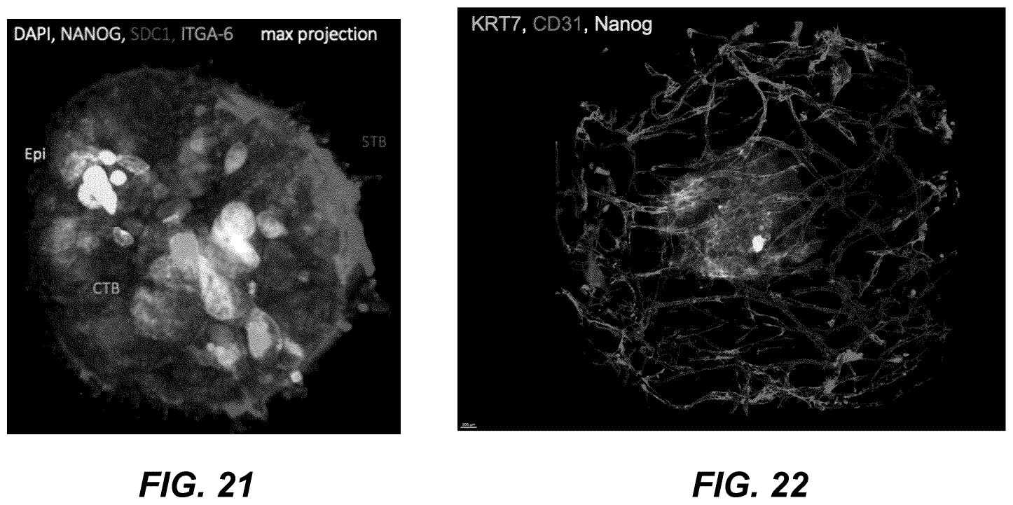

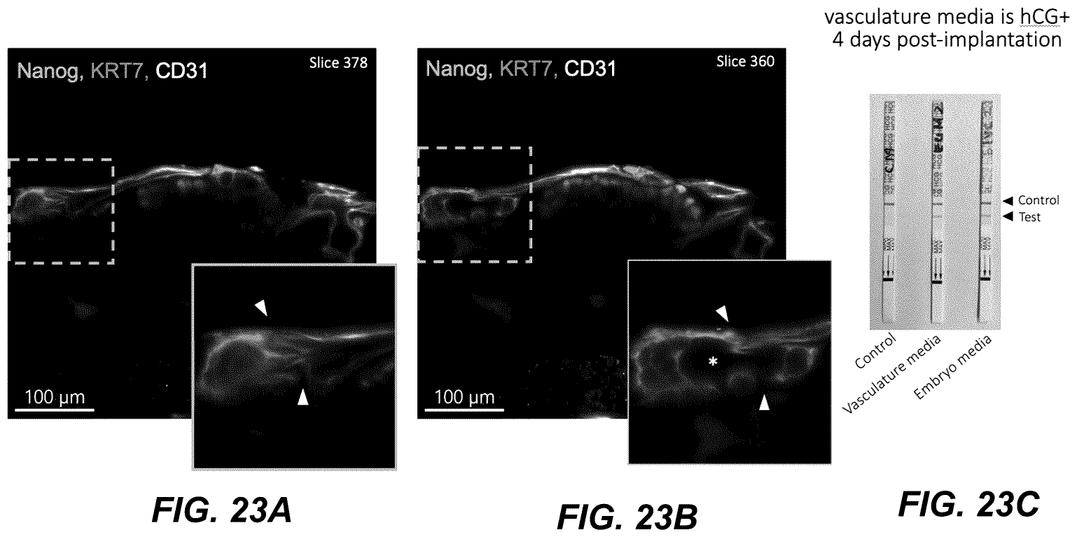

Upon 3D implantation, blastoids grew and differentiated in the device (FIG. 20A). Blastoids are collected on Day4 (D4) and cultured in the device for 4-5 days. By D7 blastoids acquire a primary yolk-sac that is lined by SOX17+ hypoblast cells (FIG. 20B). The epiblast creates cells forming an amniotic cavity and the AP2Y+ trophoblast cells proliferate and invade the hydrogel. Similar to the natural embryos, the trophoblast cells differentiated into cytotrophoblast-like cells (CTBLC) and syncytiotrophoblast-like cells (STBLC) (FIG. 21). The trophoblast cells in the blastoid were found in close proximity to the underlying CD31+ vasculature (FIGS. 22 and 23A-B). Furthermore, functional interaction between the trophoblast cells and the vasculature were measured using off-the-shelf pregnancy tests. After 4 days of implantation, both the vascular and embryo media were positive for hCG indicating that human chorionic gonadotropin (hCG) produced by the trophoblast cells were absorbed by the blood vessels and circulated in the media channels (FIG. 23C).

### Example 4

### Development of a Uterus-Like Tissue

This example describes an exemplary protocol for generating a 3-dimensional uterus-like tissue.

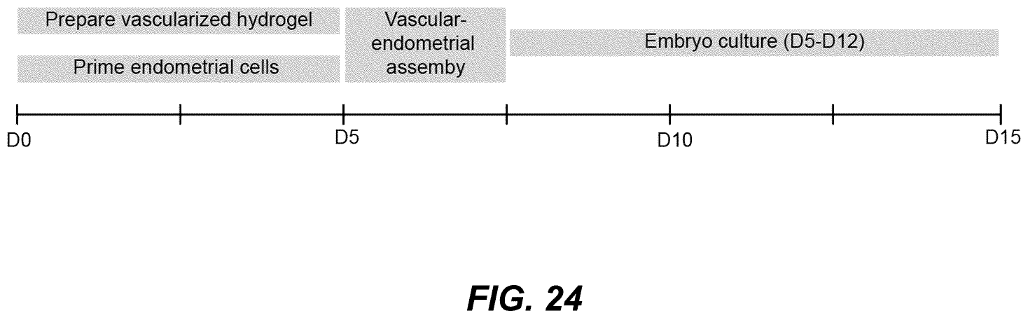

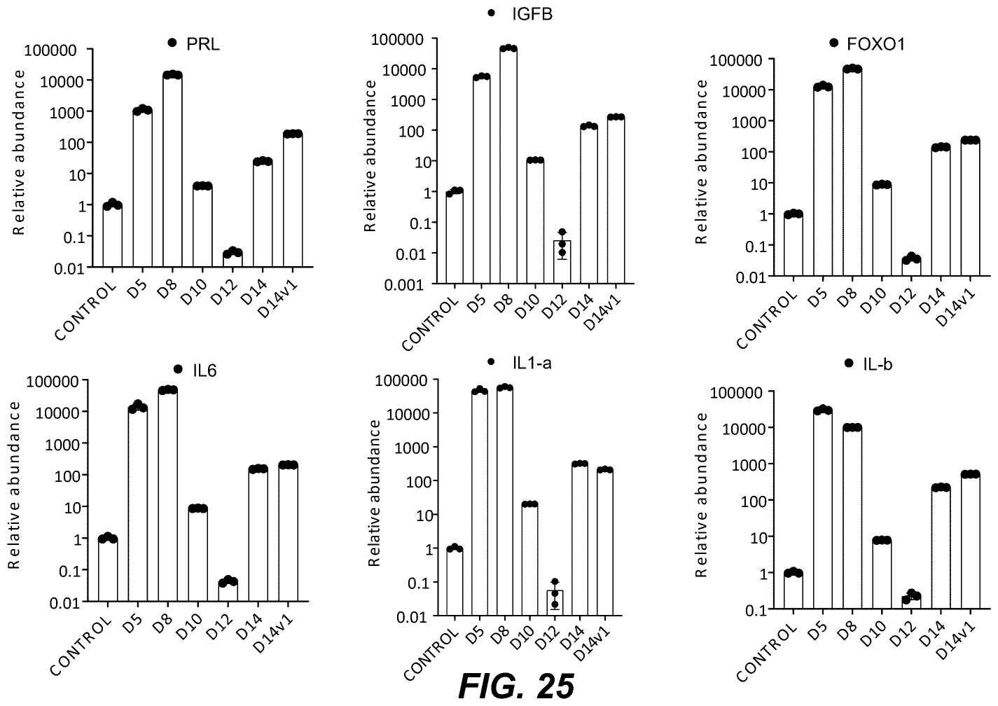

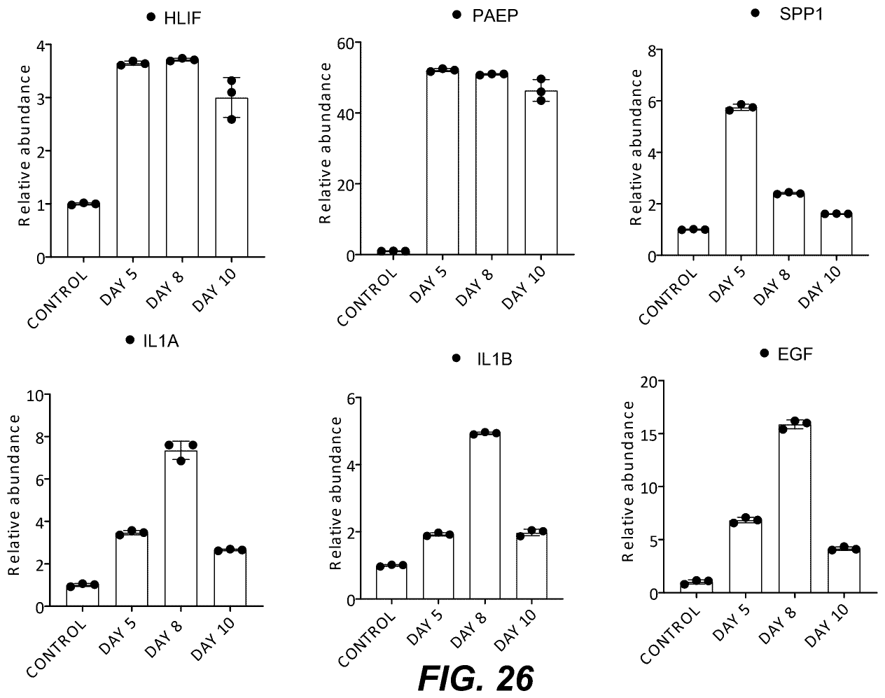

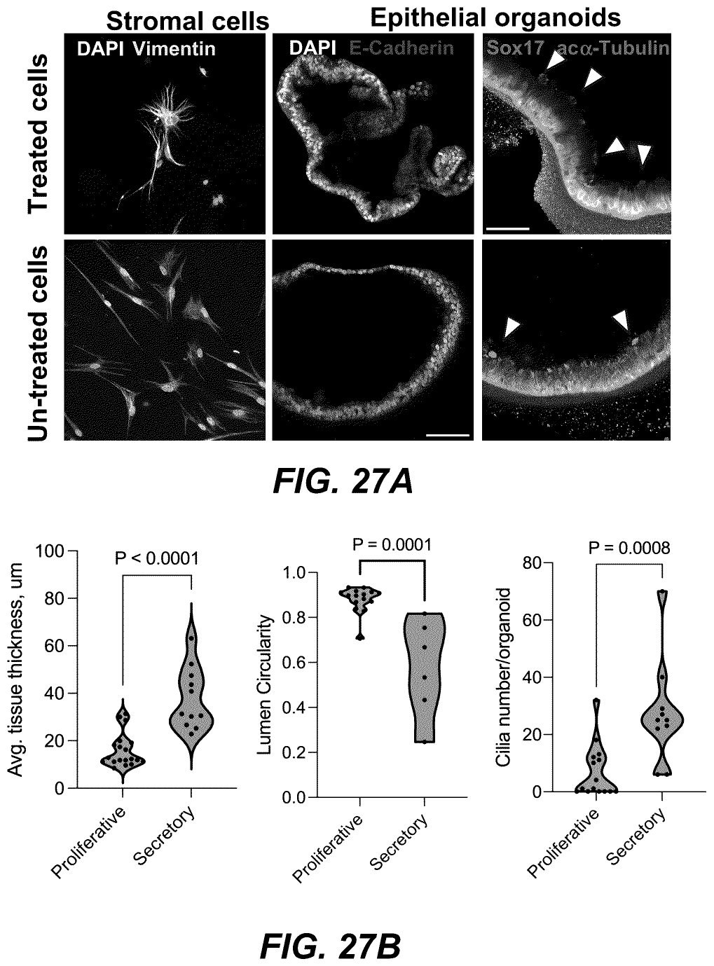

To improve upon the vascularized tissue, a 3D uterus-like tissue was generated by assembling endometrial epithelial organoids and stromal cells with pre-made vasculature. The detailed protocol is shown in FIG. 24. Endometrial epithelial organoids (EEOs) and stromal cells (ESCs) were hormonally primed using β-estradiol (E2), Medroxyprogesterone acetate (MPA) and cyclic adenosine monophosphate (cAMP), collectively called EPC. MPA is a synthetic progestin that mimics the effects of the natural hormone progesterone. Both MPA and Progesterone (P4) can be used for this protocol. While E2 helps maintain proliferation of the endometrial cells, administration of P4 helps differentiate the cells into a secretory phenotype, where they secrete cytokines, hormones and growth factors as shown in FIGS. 25 and 26. Morphological analysis shows that hormonally treated epithelial organoids transform into glandular morphology with increased tissue thickness and foldings and apical localization of acetylated alpha-tubulin (FIGS. 27A-27B). However, hormonally primed stromal cells demonstrated a rounder morphology with larger nuclear size indicating decidualization. This transformation in both cell types is reminiscent of a receptive endometrium, when it is ready for implantation also known as the ‘window of implantation’. The protocol shown in FIG. 24 is to prime EEOs and ESCs separately and then assemble them with pre-made blood vessels on D5. This ensures proper growth and transformation of vasculature and endometrial cells prior to assembly as they have different media and reagents.

Once assembled, the media on the endometrial chamber is switched to a minimum differentiation media (MDM), while maintaining the hormonal treatment of EPC. MDM is an intermediate media used for vascular endometrium before the embryo is added. MDM comprises Advanced DMEM/F12 (Fisher Scientific, 12-634-010) supplemented with 1× N2, 1× B27,1× Antibiotic-Antimycotic (Thermo Fisher, 15240062), 1× Glutamax (Thermo Fisher Scientific 35050061), 1.25 mM NAC (Sigma Aldrich, A7250-5G), 1 μM MPA (Sigma-Aldrich, 1629-1G), 0.5 mM 8-bromo-cAMP (STEM CELL TECHNOLOGIES, 73602), 1 μM B-estradiol (Sigma Aldrich, E8875-1G), and 10 μM Y27 (STEM CELL TECHNOLOGIES 72304).

To ensure compatibility of the cell types, a hydrogel composition that works for all three tissue types-vasculature, EEOs and ESCs, were developed. For the assembly 60% Fibrin (8 mg/mL), 20% Collagen-I (5 mg/mL), 10% Laminin and 10% Fibronectin were used. For the concentration of stromal cells, 1.5 Million/mL along with EEOs were used for the assembly and a 1:5 ratio of Thrombin:Fibrinogen used for polymerization of the hydrogel.

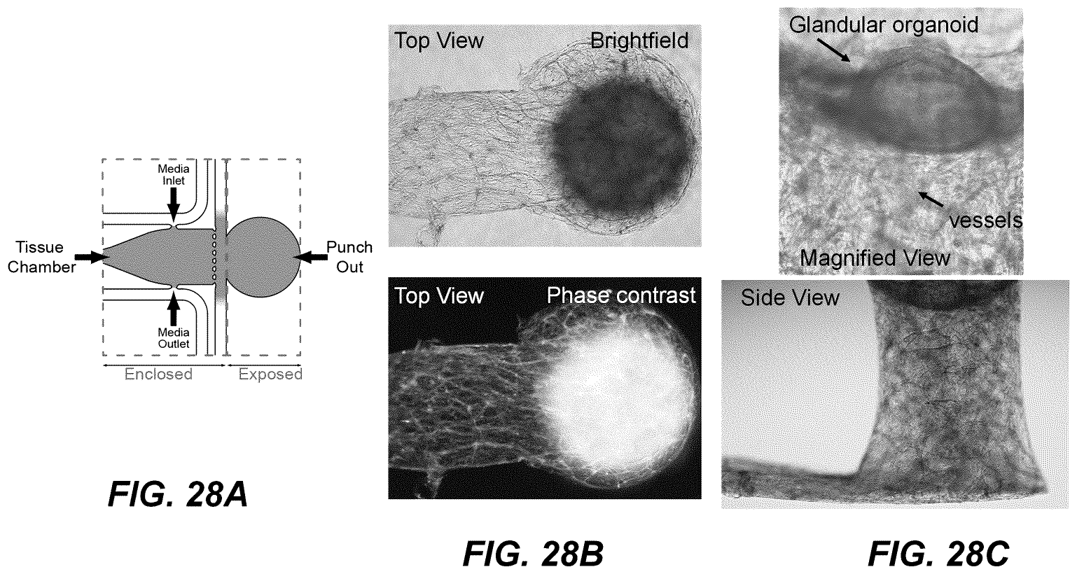

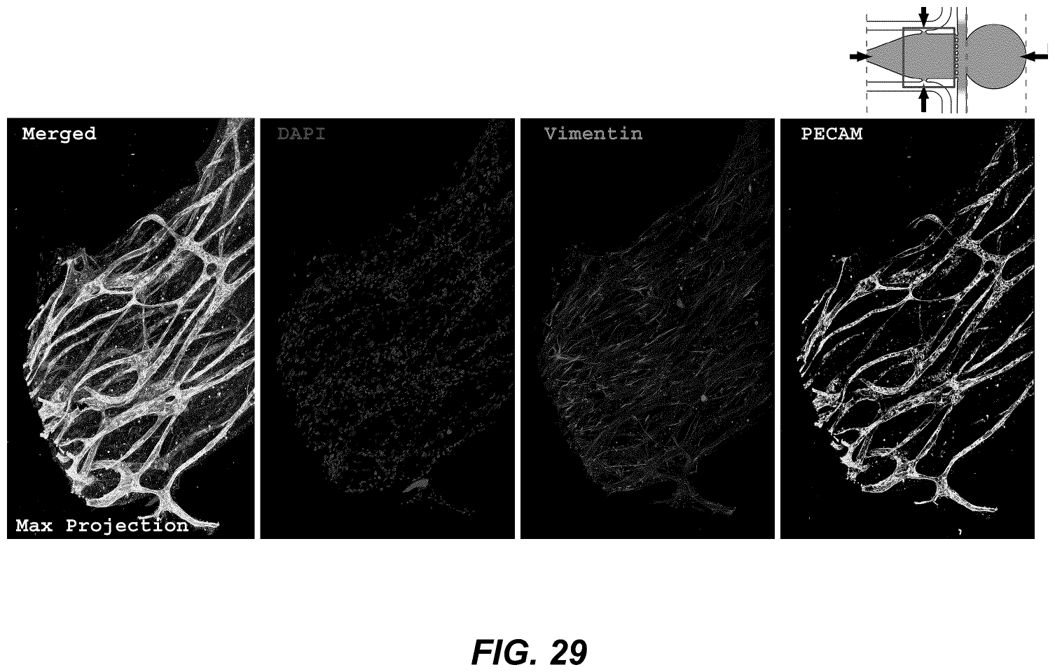

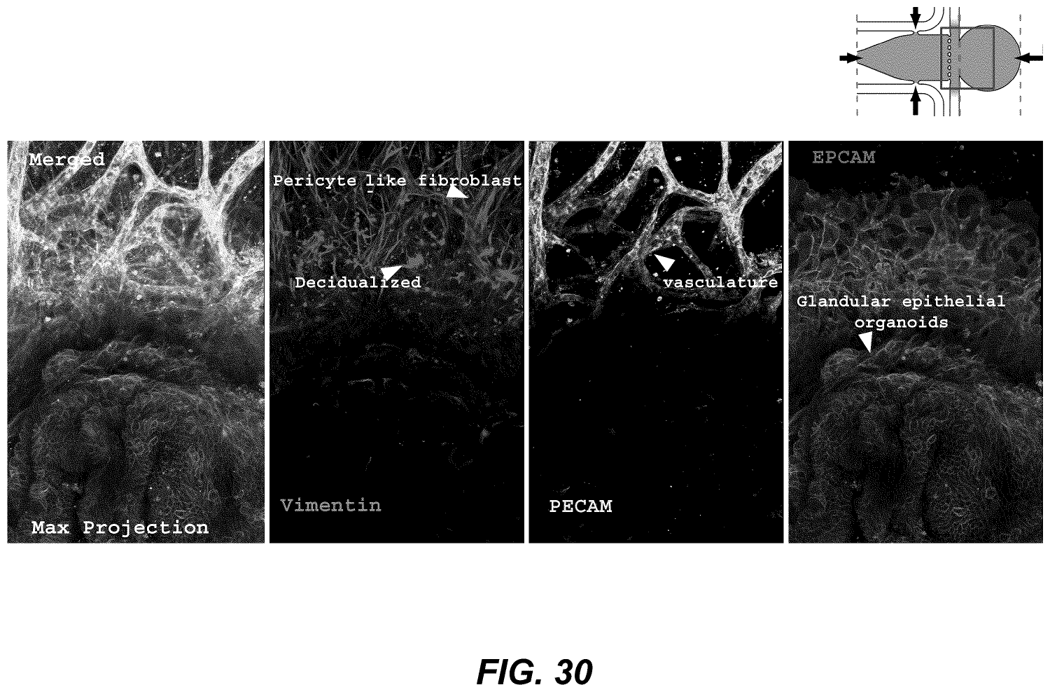

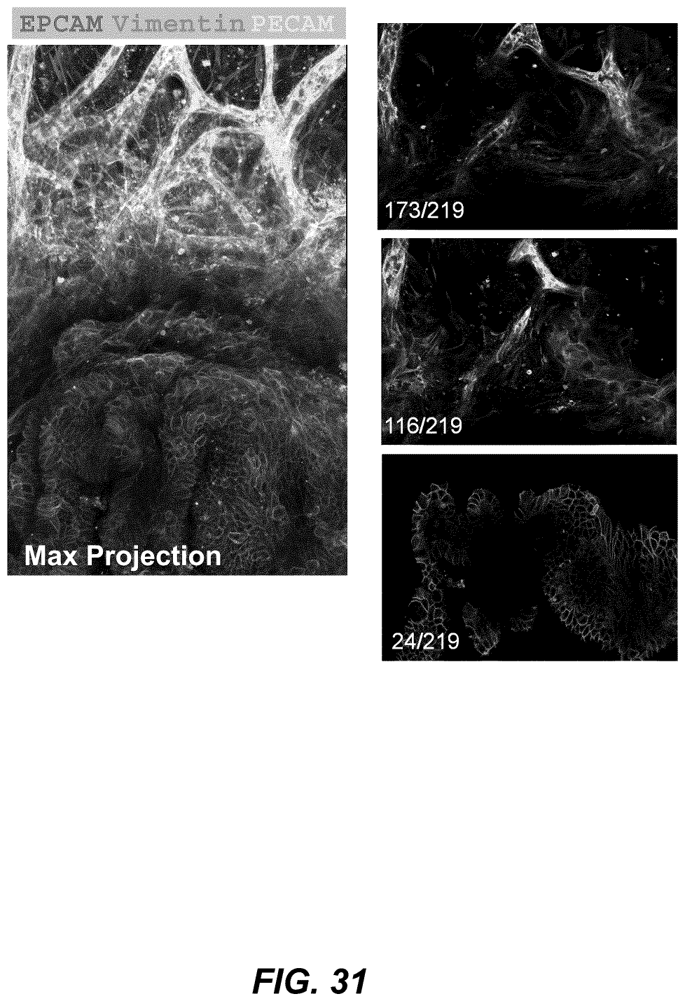

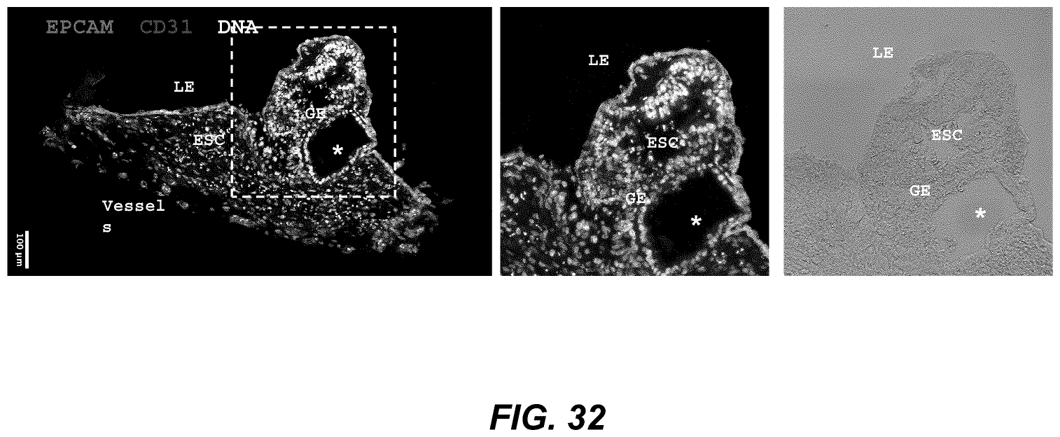

FIGS. 28B-28C show transmitted light images of a vascularized endometrial tissue dissected out of the device. This tissue exhibits a stratified layers of luminal epithelial (LE), glandular epithelia (GE), endometrial stromal cells (ESC) and endothelial blood vessels (FIGS. 29-32).

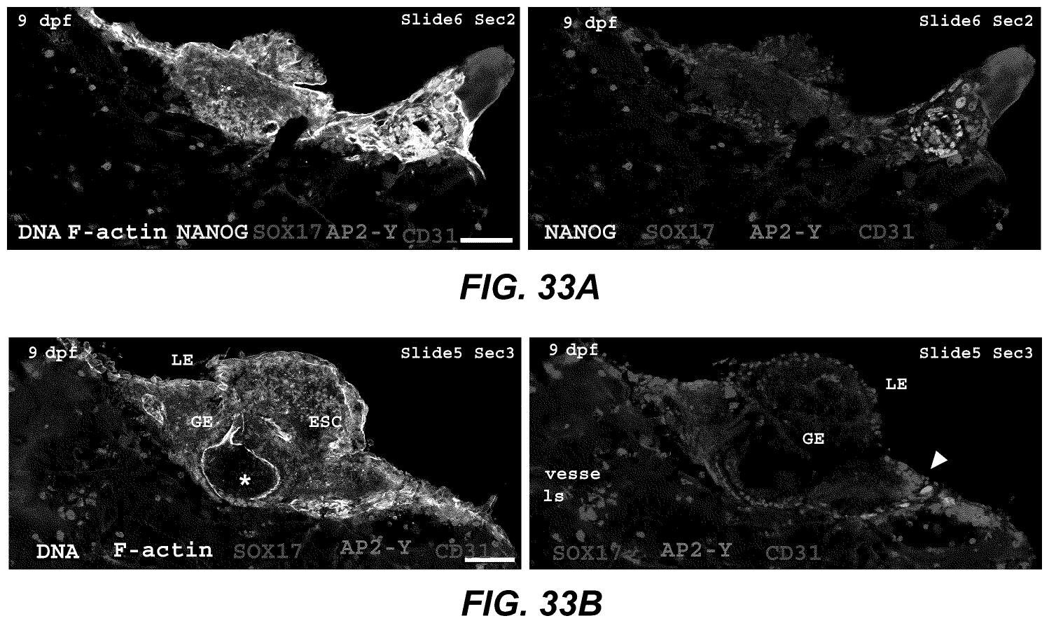

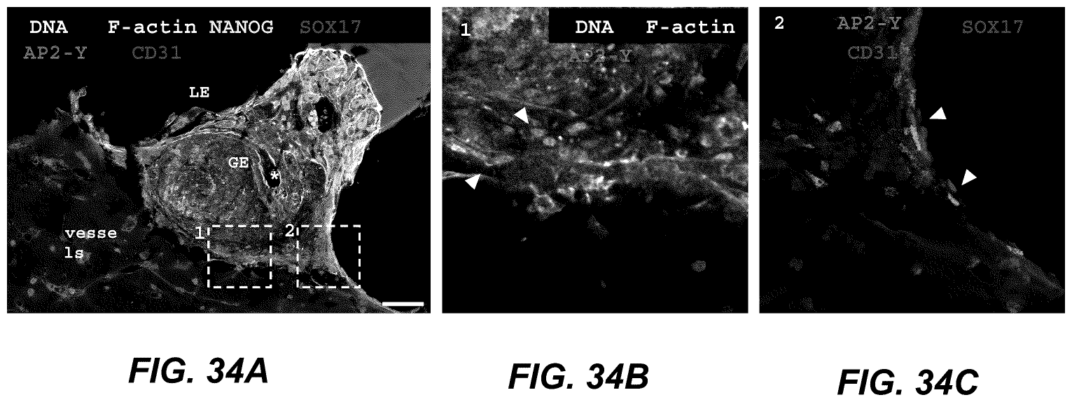

A protocol was developed to implant human embryos in this vascularized endometrial assembly (FIG. 24). FIG. 33A shows a section of a 9 dpf human embryo implanted in the vascularized endometrial platform. The image on the right shows AP2-Y+ trophoblast cells interacting with endometrial epithelial cells. FIG. 33B shows endometrial epithelial cells differentiate into luminal epithelial (LE) and glandular epithelial cells (GE). The LE cells express SOX17, and endometrial stromal cells (ESC) are present in the area around the GE. The image on the right shows trophoblast cells actively invading the tissue and interacting with LE and GE cells. Trophoblast cells migrate deep within the endometrial tissue and away from the embryo (FIGS. 34A-B) and SOX17+ LE cells encapsulate the migratory trophoblast cells (FIG. 34C).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of preparing a perfusable vascularized tissue for in vitro embryo implantation and growth, the method comprising:
co-culturing endothelial cells and fibroblast cells in a hydrogel under a condition allowing the endothelial cells and fibroblast cells to self-organize into a perfusable vascularized tissue.

2. The method of claim 1, wherein the endothelial cells comprise endothelial progenitor cells derived from stem cells, endothelial progenitor cells derived from umbzilical cord blood, human umbilical cord vascular endothelial cells, mouse brain endothelial cells, an immortalized vascular endothelial cell line, or any combination thereof; and/or wherein the fibroblast cells comprise fibroblast cells derived from stem cells, human uterine fibroblast cells, human primary brain vascular fibroblast, mouse embryonic brain vascular fibroblast, human dermal fibroblast, human perivascular fibroblast, human lung fibroblast, or any combination thereof.

3. The method of claim 2, wherein the immortalized vascular endothelial cell line is selected from the group consisting of: EA.hy926, HMEC-1, TIME cells, CI-huMEC, EC-RF24, iMAEC, and a combination thereof.

4. (canceled)

5. The method of claim 1, wherein the endothelial cells comprise human umbilical cord vascular endothelial cells and/or the fibroblast cells comprise human lung fibroblast cells.

6. (canceled)

7. The method of claim 1, wherein the hydrogel comprises collagen, hyaluronic acid, polyethylene glycol, polyacrylamide, or a combination thereof.

8. The method of claim 1, wherein the hydrogel is fibrin, optionally the fibrin is fibrinogen cross-linked with thrombin at a thrombin: fibrinogen ratio of about 1:5.

9. The method of claim 1, wherein the hydrogel is functionalized with one or more extracellular matrix component, optionally the extracellular matrix component comprises laminin, fibronectin, collagen-III, collagen-IV, collagen-V, collagen-I, hyaluronic acid, or any combination thereof.

10. The method of claim 1, wherein the hydrogel comprises about 40-60% fibrin, about 10-20% collagen-I, about 5-10% laminin, and about 5-10% fibronectin; and optionally the fibrin comprises thrombin and fibrinogen at a ratio of about 1:5.

11. The method of claim 1, wherein the culturing comprises adding a extracellular matrix solution to a solidified hydrogel mixture comprising the endothelial cells and fibroblast cells and incubating for a time period, optionally the incubation is for about 15 minutes, and replacing the extracellular matrix solution with a vasculature media, optionally, the vasculature media is endothelial cell and fibroblast growth medium.

12. The method of claim 11, wherein a hydrostatic gradient of the vasculature media is maintained to generate a media flow from a higher pressure area to a lower pressure area.

13. The method of claim 11, wherein the vasculature media comprises fetal bovine serum, hydrocortisone, human fibroblast growth factor-B (hFGF-B), vascular endothelial growth factor (VEGF), R3-insulin-like growth factor-1 (R3-IGF-1), ascorbic acid, heparin, human epidermal growth factor (hEGF), an antimicrobial, or any combination thereof.

14. (canceled)

15. (canceled)

16. (canceled)

17. (canceled)

18. (canceled)

19. The method of claim 1, wherein the co-culturing is for at least about 5-6 days, optionally until the vasculature fuses with fluidic channels or the bioreactor to create a perfusable network.

20. (canceled)

21. (canceled)

22. The method of claim 1, comprising co-culturing endometrial cells, placental cells, trophoblast cells, trophoblast organoids, or any combination thereof in the hydrogel.

23. A perfusable vascularized tissue for in vitro mammalian embryo or stem-cell derived embryo model implantation and culturing obtainable or obtained from the method of claim 1.

24. (canceled)

25. A method of preparing a uterus-like tissue for in vitro embryo implantation and culturing, the method comprising:
preparing a perfusable vascularized tissue according to the method of claim 1,
priming endometrial epithelial organoids, stromal fibroblast cells, immune cells with one or more hormones, and
assembling the primed endometrial epithelial organoids, primed stromal fibroblast cells endothelial cells, fibroblast and immune cells with the perfusable vascularized tissue to form a uterus-like tissue.

26. The method of claim 25, wherein the one or more hormones comprise β-estradiol (E2), Medroxyprogesterone acetate (MPA), Progesterone (P4), cyclic adenosine monophosphate (cAMP), or any combination thereof.

27. (canceled)

28. The method of claim 25, wherein the assembling comprises culturing the primed endometrial epithelial organoids and primed stromal cells in the perfusable vascularized tissue in a minimal differentiation media in the presence of the one or more hormones, wherein the minimal differentiation media comprises a basal culture media supplemented with N-acetylcysteine, Oestradiol, a cyclic AMP analogue, optionally, 8-bromo-cAMP and a progestin, optionally, medroxyprogesterone acetate (MPA).

29. (canceled)

30. (canceled)

31. (canceled)

32. The method of claim 25, wherein preparing the perfusable vascularized tissue and/or the assembling occurs in a microfluidic device comprising at least one tissue chamber, wherein the at least one tissue chamber comprises an enclosed chamber, an exposed chamber accessible from a top plate via an opening, and interdigitated pillars at the interface between the enclosed chamber and the exposed chamber, the enclosed chamber in fluidic communication with the enclosed chamber, and
wherein the enclosed chamber is fluidly connected to a media inlet and a media outlet, and the enclosed chamber and the exposed chamber each is connected to at least one hydrogel loading port,
wherein preparing the perfusable vascularized tissue further comprises (1) seeding the endothelial cells, the fibroblast cells, and the hydrogel in the enclosed chamber and in the exposed chamber via different ports, thereby creating a continuous solution in both the enclosed and exposed chambers; and/or (2) adding an extracellular matrix solution and/or a vasculature media to a solidified hydrogel mixture via the media inlet and/or the media outlet.

33. The method of claim 32, further comprising loading the primed endometrial epithelial organoids and primed stromal cells to the tissue chamber containing the perfusable vascularized tissue through the opening in the exposed chamber.

34. (canceled)

35. A uterus-like tissue for in vitro mammalian embryo or stem-cell derived embryo model implantation and culturing obtainable by the method of claim 25.

36.-77. (canceled)
